(19) United States
(12) Patent Application Publication
(10) Pub. No.: US 2019/0022209 A1
(43) Pub. Date: Jan. 24, 2019

(54) METHODS AND COMPOSITIONS FOR INFLUENZA VACCINATION

(71) Applicant: ETUBICS CORPORATION, Seattle, WA (US)

(72) Inventors: Frank R. Jones, Seattle, WA (US); Joseph Balint, Seattle, WA (US); Elizabeth Gabitzsch, Seattle, WA (US); Yvette Latchman, Seattle, WA (US); Adrian Rice, Seattle, WA (US)

(21) Appl. No.: 16/070,104

(22) PCT Filed: Jan. 13, 2017
(86) PCT No.: PCT/US2017/013480

§ 371 Date: Jul. 13, 2018

Related U.S. Application Data
(60) Provisional application No. 62/294,840, filed on Feb. 12, 2016.
(60) Provisional application No. 62/279,267, filed on Jan. 15, 2016.

(51) Int. Cl. A61K 39/145

(52) U.S. Cl. CPC A61K 39/145; A61K 2039/5256

(57) ABSTRACT

Methods for constructing and producing a recombinant adenovirus based vector vaccine containing multiple influenza antigen genes for use in generating broad based immune responses against influenza A and B viruses and that allows for multiple vaccinations in individuals with preexisting immunity to adenovirus are described. Specifically, the recombinant adenovirus based vector is a replication defective adenovirus vector comprising a deletion in an early 2b (E2b) gene.

### CROSS REFERENCE

This application claims the benefit of U.S. Provisional Patent Application No. 62/279,267, filed Jan. 15, 2016, and U.S. Provisional Patent Application No. 62/294,840, filed Feb. 12, 2016, the entire contents of which are incorporated by reference.

### STATEMENT OF GOVERNMENT INTEREST

This invention was made with government support under research grant RO1AI111364 awarded by the National Institutes of Health (NIH), National Institutes of Allergy and Infectious Diseases (NIAID). The government may have certain rights in this invention.

### BACKGROUND

Vaccines help the body fight disease by training the immune system to recognize and destroy harmful substances and diseased cells. Vaccines can be largely grouped into two types, preventive and treatment vaccines. Prevention vaccines are given to healthy people to prevent the development of specific diseases, while treatment vaccines, also referred to as immunotherapies, are given to a person who has been diagnosed with disease to help stop the disease from growing and spreading or as a preventive.

Viral vaccines are currently being developed to help fight infectious diseases and cancers. These viral vaccines work by inducing expression of a small fraction of genes associated with a disease within the host's cells, which in turn, enhance the host's immune system to identify and destroy diseased cells. As such, clinical response of a viral vaccine can depend on the ability of vaccine to obtain a high level immunogenicity and have sustained long-term expression.

There remains a need for developing methods and compositions for enhanced therapeutic response to complex diseases such as infectious diseases.

### SUMMARY

In various aspects, the present disclosure provides a composition comprising: a replication defective adenovirus vector comprising a deletion in an E2b gene region; and a nucleic acid sequence encoding an influenza A target antigen and an influenza B target antigen.

In some aspects, the influenza A target antigen is a target antigen of an influenza virus A. In further aspects, the influenza A target antigen and the influenza B target antigen are target antigens common to an influenza virus A and an influenza virus B.

In some aspects, said replication defective adenovirus vector further comprises a deletion in an E1 region. In further aspects, said replication defective adenovirus vector further comprises a deletion in an E3 region. In still further aspects, said replication defective adenovirus vector further comprises a deletion in an E4 region. In some aspects, said replication defective adenovirus vector further comprises a deletion in an E3 and an E4 region.

In some aspects, the influenza A target antigen comprises an antigen of a virus selected from the group consisting of H3N2, H9N1, H1N1, H2N2, H7N7, H1N2, H9N2, H7N2, H7N3, H10N7, and combinations thereof. In other aspects, the influenza B target antigen comprises antigens of a virus selected from the influenza B/Yamagata and influenza B/Victoria viruses.

In other aspects, the influenza A target antigen is an antigen from a protein selected from the group consisting of matrix protein M2, the M2e portion of matrix protein M2, hemagglutinin, hemagglutinin stalk, neuraminidase, nucleoprotein, matrix protein M1, and combinations thereof. In some aspects, the influenza B target antigen is an antigen from a protein selected from the group consisting of BM2 protein, hemagglutinin, hemagglutinin stalk, neuraminidase, nucleoprotein, and combinations thereof.

In some aspects, the deletion comprises a base pair. In further aspects, the deletion comprises at least 20, at least 30, at least 40, at least 50, at least 60, at least 70, at least 80, at least 90, at least 100, at least 110, at least 120, at least 130, at least 140, or at least 150 base pairs.

In still further aspects, the deletion comprises more than 150, more than 160, more than 170, more than 180, more than 190, more than 200, more than 250, or more than 300 base pairs.

In some aspects, the adenovirus vector comprises nucleic acids encoding at least one, at least 2, at least 3, at least 4, at least 5, at least 6, at least 7, at least 8, at least 9 or at least 10 influenza A target antigens. In some aspects, the adenovirus vector comprises nucleic acids encoding a plurality of influenza A target antigens. In other aspects, the adenovirus vector comprises nucleic acids encoding at least one, at least 2, at least 3, at least 4, at least 5, at least 6, at least 7, at least 8, at least 9 or at least 10 influenza B target antigens. In some aspects, the adenovirus vector comprises nucleic acids encoding a plurality of influenza B target antigens.

In further aspects, the adenovirus vector further comprises an element to increase the expression of the influenza A target antigen, the influenza B target antigen, or both. In some aspects, said element comprises at least one element, at least 2 elements, at least 3 elements, at least 4 elements, or at least 5 elements. In some aspects, said element comprises an internal ribosome binding site. In some aspects, said element comprises a constitutive promotor. In other aspects, said element comprises an inducible promotor.

In other aspects, said element comprises a transcription enhancer. In some aspects, said transcription enhancer is a Rous sarcoma virus (RSV) enhancer. In some aspects, said element does not contain a palindromic sequence.

In some aspects, the adenovirus vector further comprises nucleic acid sequences that encode proteins that increase the immunogenicity of the influenza A target antigen, the influenza B target antigen, or both. In some aspects, the adenovirus vector is not a gutted vector. In some aspects, the composition or the replication-defective adenovirus vector further comprises a nucleic acid sequences encoding a costimulatory molecule. In further aspects, the costimulatory molecule comprises B7, ICAM-1, LFA-3, or a combination thereof. In still further aspects, the costimulatory molecule comprises a combination of B7, ICAM-1, and LFA-3. In some aspects, the adenovirus vector comprises the nucleic acid sequence encoding an influenza A target antigen and an influenza B target antigen. In some embodiments, the composition comprises at least 1×10⁸ viral particles (VPs) and not more than 5×10¹⁰ VPs. In other embodiments, the composition comprises at least 1×10⁸ viral particles (VPs) and not more than 1×10¹² VPs.

In various aspects, the present disclosure provides a method of generating an immune response against an influenza A target antigen and an influenza B target antigen in an individual in need thereof, comprising administering to the individual a composition according to any of the above described compositions.

In various aspects, the present disclosure provides a method of generating an immune response against an influenza A target antigen and an influenza B target antigen in an individual comprising administering to the individual a first adenovirus vector comprising: a replication defective adenovirus vector, wherein the adenovirus vector has a deletion in the E2b region, and a nucleic acid encoding an influenza A target antigen and an influenza B target antigen; administering to the individual a second adenovirus vector comprising: (a) a replication defective adenovirus vector, wherein the adenovirus vector has a deletion in the E2b region, and (b) nucleic acids encoding an influenza A target antigen and an influenza B target antigen; thereby generating an immune response against one or more influenza A and B target antigens.

In various aspects, the present disclosure provides a method of generating an immune response against an influenza A target antigen and an influenza B target antigen in an individual comprising: (a) administering to the individual a first vector comprising: (i) a replication defective adenovirus vector, wherein said adenovirus vector has a deletion in the E2b region, and (ii) a nucleic acid encoding a first influenza A target antigen and a first influenza B target antigen; and (b) subsequently administering to the individual a second vector comprising: (i) the replication defective adenovirus vector of step (a), and (ii) a nucleic acid encoding a second influenza A target antigen and a second influenza B target antigen, wherein the second influenza A target antigen of the second vector is the same or different from the first influenza A target antigen of the first vector, and wherein the second influenza B target antigen of the second vector is the same or different from the first influenza B target antigen of the first vector; thereby generating an immune response against the first target antigen and the second target antigen.

In various aspects, the present disclosure provides a method of generating an immune response against an influenza A target antigen and an influenza B target antigen in an individual comprising: administering to the individual an adenovirus vector comprising a replication defective adenovirus vector, wherein the adenovirus vector has a deletion in the E2b region and nucleic acids encoding an influenza A target antigen and an influenza B target antigen; and re-administering the adenovirus vector at least once to the individual; thereby generating an immune response against the influenza A and B target antigens.

In various aspects, the present disclosure provides a method of constructing a universal influenza vaccine vector comprising inserting nucleic acids encoding an influenza A target antigen and an influenza B target antigen into a replication defective adenovirus vector, wherein the adenovirus vector has a deletion in the E2b region.

In some aspects, the influenza A target antigen comprises an antigen of a virus selected from the group consisting of H3N2, H9N1, H1N1, H2N2, H7N7, H1N2, H9N2, H7N2, H7N3, H10N7, and combinations thereof. In some aspects, the influenza B target antigen comprises an antigen of a virus selected from the influenza B/Yamagata and influenza B/Victoria viruses.

In other aspects, the influenza A target antigen is an antigen from a protein selected from the group consisting of matrix protein M2, the M2e portion of matrix protein M2, hemagglutinin, hemagglutinin stalk, neuraminidase, nucleoprotein, matrix protein M1, and combinations thereof. In still other aspects, the influenza B target antigen is an antigen from a protein selected from the group consisting of BM2 protein, hemagglutinin, hemagglutinin stalk, neuraminidase, nucleoprotein, and combinations thereof.

In some aspects, the individual has preexisting immunity to adenovirus. In some aspects, the adenovirus vector is not a gutted vector.

In other aspects, a first vector is not a gutted vector. In further aspects, a second vector is not a gutted vector. In still further aspects, the first and second adenovirus vectors are not gutted vectors. In some aspects, the individual has preexisting immunity to adenovirus 5.

In some aspects, the first and second target antigens of the first and the second vectors are derived from the same infectious organism. In other aspects, the first and second target antigens of the first and the second vectors are derived from different infectious organisms. In some aspects, the influenza A target antigen and the influenza B target antigen are different target antigens.

In some aspects, the influenza A target antigen is a target antigen of an influenza virus A. In some aspects, the influenza A target antigen and the influenza B target antigen are target antigens common to an influenza virus A and an influenza virus B.

In some aspects, said replication defective adenovirus vector further comprises a deletion in an E1 region. In further aspects, said replication defective adenovirus vector further comprises a deletion in an E3 region. In still further aspects, said replication defective adenovirus vector further comprises a deletion in an E4 region. In still further aspects, said replication defective adenovirus vector further comprises a deletion in an E3 and an E4 region.

In some aspects, the deletion comprises a base pair. In further aspects, the deletion comprises at least 20, at least 30, at least 40, at least 50, at least 60, at least 70, at least 80, at least 90, at least 100, at least 110, at least 120, at least 130, at least 140, or at least 150 base pairs.

In still further aspects, the deletion comprises more than 150, more than 160, more than 170, more than 180, more than 190, more than 200, more than 250, or more than 300 base pairs.

In some aspects, the adenovirus vector comprises nucleic acid sequences encoding at least one, at least 2, at least 3, at least 4, at least 5, at least 6, at least 7, at least 8, at least 9 or at least 10 influenza A and B target antigens.

In some aspects, the adenovirus vector further comprises an element to increase the expression of the influenza A and influenza B target antigen. In some aspects, said element comprises at least one element, at least 2 elements, at least 3 elements, at least 4 elements, or at least 5 elements. In some aspects, said element comprises an internal ribosome binding site. In other aspects, said element comprises a constitutive promotor. In some aspects, said element comprises an inducible promotor. In other aspects, said element comprises a transcription enhancer. In further aspects, said transcription enhancer is a Rous sarcoma virus (RSV) enhancer. In some aspects, said element does not contain a palindromic sequence.

In some aspects, the adenovirus vector further comprises a nucleic acid sequence that encodes a polypeptide that increases the immunogenicity of the influenza A target antigen, the influenza B target antigen, or both. In some aspects, the influenza A target antigen comprises M and the influenza B target antigen comprises BM2.

In other aspects, the influenza A target antigen, the influenza B target antigen, or both comprise hemagglutinin. In some aspects, the hemagglutinin comprises an HAI domain. In some aspects, herein the hemagglutinin comprises an HA2 domain. In other aspects, herein the hemagglutinin comprises a stalk domain. In some aspects, the influenza A target antigen, the influenza B target antigen, or both comprise a neuraminidase.

In other aspects, the influenza A target antigen, the influenza B target antigen, or both comprise a nucleoprotein (NP). In still other aspects, the influenza A target antigen comprises matrix protein Ml. In some aspects, the influenza A target antigen comprises matrix protein M2. In other aspects, the influenza A target antigen comprises matrix protein M2e. In some aspects, the influenza A target antigen, the influenza B target antigen, or both are encoded by a nucleic acid sequence with at least 60%, 70%, 75%, 80%, 85%, 90%, 95%, 97%, 99%, 99.5%, or 100% sequence identity to a sequence encoding a BM2 protein, a hemagglutinin, a hemagglutinin stalk, a neuraminidase, a nucleoprotein, a matrix protein M1, a matrix protein M2 or any combination thereof. In some embodiments, the method comprises administering at least 1×10⁸ viral particles (VPs) and not more than 5×10¹⁰ VPs. In other embodiments, the method comprises administering at least 1×10⁸ viral particles (VPs) and not more than 1×10¹² VPs.

Embodiments discussed in the context of methods and/or compositions described herein may be employed with respect to any other method or composition described herein. Thus, an embodiment pertaining to one method or composition may be applied to other methods and compositions as well.

Other objects, features and advantages will become apparent from the following detailed description. It should be understood, however, that the detailed description and the specific examples, while indicating particular embodiments, are given by way of illustration only, since various changes and modifications within the spirit and scope of the invention will become apparent to those skilled in the art from this detailed description.

### BRIEF DESCRIPTION OF THE DRAWINGS

FIG. 1 illustrates schematic diagrams of multiple gene constructs of the present disclosure.

FIG. 1A illustrates a triple gene insert containing a matrix 1 (M1) protein, nucleoprotein (NP) protein, hemagglutinin (HA) of influenza A, and a Gly-Ser-Gly linker between each protein gene to be used for insertion into Ad5 [E1-, E2b-].

FIG. 1B illustrates an Ad5 [E1-, E2b-] containing two antigen gene sequences separated by a single “self-cleaving” 2A peptide derived from the Porcine teschovirus-1 and Thosea asigna virus, respectively.

FIG. 2 illustrates a Western blot expression of influenza M1, NP, and HA antigens in a single Ad5 [E1-, E2b-]-based platform.

FIG. 3 illustrates a graph demonstrating generation of antibody responses to HA antigen after immunizations with escalating doses of an Ad5 [E1-, E2b-]-M1/NP/HA vaccine but not with an Ad5 [E1-, E2b-]-null empty control vector. Values are Mean +/−SEM.

FIG. 4 illustrates a graph demonstrating generation of cell-mediated immunity (CMI) responses as determined by ELISpot assays for IFN-secreting splenocytes to M1, NP, and HA antigen after immunizations with escalating doses of an Ad5 [E1-, E2b-]-M1/NP/HA vaccine but not with an Ad5 [E1-, E2b-]-null empty control vector. Specificity of ELISpot responses was shown by lack of splenocyte reactivity with irrelevant SIV-Nef and SIV-Vif peptide pools. Values are Mean +/−SEM.

FIG. 5 is a graph demonstrating generation of cytolytic T lymphocyte (CTL) responses as determined by ELISpot assays for granzyme B secreting splenocytes to M1, NP, and HA antigen after immunizations with escalating doses of an Ad5 [E1-, E2bd-]-M1/NP/HA vaccine but not with an Ad5 [E1-, E2b-]-null empty control vector. Specificity of ELISpot responses was shown by lack of splenocyte reactivity with irrelevant SIV-Nef and SIV-Vif peptide pools. Values are Mean +/−SEM.

FIG. 6 illustrates a graph demonstrating generation of CMI responses in a time course (longitudinal) study as determined by ELISpot assays for IFN-γ-secreting splenocytes to NP and HA antigen after immunizations with an Ad5 [E1-, E2b-]-M1/NP/HA vaccine once (red line; Group 1), twice 2-weeks apart (blue line; Group 2), twice 1-month apart (green line; Group 3), or twice 2-months apart (orange line; Group 4). Values are Mean +/−SD. Arrows indicate immunization times.

FIG. 7 illustrates a graph demonstrating generation of antibody (Ab) responses to HA antigen in a time course (longitudinal) study after immunizations with an Ad5 [E1-, E2b-]-M1/NP/HA vaccine once (red line; Group 1), twice 2-weeks apart (blue line; Group 2), twice 1-month apart (green line; Group 3), or twice 2-months apart (orange line; Group 4). Values are Mean +/−SD. Arrows indicate immunization times.

FIG. 8 illustrates quantitation of the Influenza-A or Influenza-B HA antibody response in serum as determined by an enzyme-linked immunosorbent assay (ELISA) after immunization in mice with Ad5 [E1-, E2b-] influenza vaccines.

FIG. 8A illustrates quantification of the Influenza-A HA antibody response.

FIG. 8B illustrates quantification of the Influenza-B HA antibody response.

FIG. 9 illustrates the cell-mediated immune response as measured by quantification of IFN-γ-expressing effector T lymphocytes in restimulated splenocytes from mice that have been immunized with a combination of an Ad5 [E1-, E2b-]-InfA-HA/M2e vaccine and an Ad5 [E1-, E2b-]-InfB-HA vaccine.

FIG. 9A illustrates the percentage of IFN-γ-expressing CD8+ splenocytes.

FIG. 9B illustrates the percentage of IFN-γ-expressing CD4+ splenocytes.

FIG. 10 illustrates the cell-mediated immune response as measured by quantification of cytokine secreting restimulated splenocytes from mice that have been immunized with a combination of an Ad5 [E1-, E2b-]-InfA-HA/M2e vaccine and an Ad5 [E1-, E2b-]-InfB-HA vaccine.

FIG. 10A illustrates quantification of IFN-γ-secreting splenocytes.

FIG. 10B illustrates quantification of IL-2-secreting splenocytes.

FIG. 11 illustrates a survival curve from the challenge study in mice immunized with an Ad5 [E1-, E2b-]-M1/NP/InfA-HA vaccine as compared with a control (null) vaccine over a period of a 60 days.

### DETAILED DESCRIPTION

The following passages describe different aspects of the invention in greater detail. Each aspect of the invention may be combined with any other aspect or aspects of the invention unless clearly indicated to the contrary. In particular, any feature indicated as being preferred or advantageous may be combined with any other feature of features indicated as being preferred or advantageous.

As used herein, unless otherwise indicated, the article “a” means one or more unless explicitly otherwise provided for.

As used herein, unless otherwise indicated, terms such as “contain,” “containing,” “include,” “including,” and the like mean “comprising.”

As used herein, unless otherwise indicated, the term “or” can be conjunctive or disjunctive.

As used herein, unless otherwise indicated, any embodiment can be combined with any other embodiment.

As used herein, unless otherwise indicated, some inventive embodiments herein contemplate numerical ranges. A variety of aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range as if explicitly written out. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. When ranges are present, the ranges include the range endpoints.

The term “adenovirus” or “Ad” refers to a group of non-enveloped DNA viruses from the family Adenoviridae. In addition to human hosts, these viruses can be found in, but are not limited to, avian, bovine, porcine and canine species. The use of any adenovirus from any of the four genera of the family Adenoviridae (e.g., Aviadenovirus, Mastadenovirus, Atadenovirus and Siadenovirus) may be contemplated as the basis of an E2b deleted virus vector, or vector containing other deletions as described herein. In addition, several serotypes are found in each species. Ad also pertains to genetic derivatives of any of these viral serotypes, including but not limited to, genetic mutation, deletion or transposition of homologous or heterologous DNA sequences.

A “helper adenovirus” or “helper virus” refers to an Ad that can supply viral functions that a particular host cell cannot (the host may provide Ad gene products such as El proteins). This virus is used to supply, in trans, functions (e.g., proteins) that are lacking in a second virus, or helper dependent virus (e.g., a gutted or gutless virus, or a virus deleted for a particular region such as E2b or other region as described herein); the first replication-incompetent virus is said to “help” the second, helper dependent virus thereby permitting the production of the second viral genome in a cell.

The term “Adenovirus5 null (Ad5null)”, as used herein, refers to a non-replicating Ad that does not contain any heterologous nucleic acid sequences for expression.

The term “First Generation adenovirus”, as used herein, refers to an Ad that has the early region 1 (E1) deleted. In additional cases, the nonessential early region 3 (E3) may also be deleted.

The term “gutted” or “gutless”, as used herein, refers to an adenovirus vector that has been deleted of all viral coding regions.

The term “transfection” as used herein refers to the introduction of foreign nucleic acid into eukaryotic cells. Transfection may be accomplished by a variety of means known to the art including calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

The term “stable transfection” or “stably transfected” refers to the introduction and integration of foreign nucleic acid, DNA or RNA, into the genome of the transfected cell. The term “stable transfectant” refers to a cell which has stably integrated foreign DNA into the genomic DNA.

The term “reporter gene” indicates a nucleotide sequence that encodes a reporter molecule (including an enzyme). A “reporter molecule” is detectable in any of a variety of detection systems, including, but not limited to enzyme-based detection assays (e.g., ELISA, as well as enzyme-based histochemical assays), fluorescent, radioactive, and luminescent systems.

In one embodiment, the E. coli β-galactosidase gene (available from Pharmacia Biotech, Pistacataway, N.J.), green fluorescent protein (GFP) (commercially available from Clontech, Palo Alto, Calif.), the human placental alkaline phosphatase gene, the chloramphenicol acetyltransferase (CAT) gene or other reporter genes that are known to the art may be employed.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The nucleic acid sequence thus codes for the amino acid sequence.

The term “heterologous nucleic acid sequence”, as used herein, refers to a nucleotide sequence that is ligated to, or is manipulated to become ligated to, a nucleic acid sequence to which it is not ligated in nature, or to which it is ligated at a different location in nature. Heterologous nucleic acid may include a nucleotide sequence that is naturally found in the cell into which it is introduced or the heterologous nucleic acid may contain some modification relative to the naturally occurring sequence.

The term “transgene” refers to any gene coding region, either natural or heterologous nucleic acid sequences or fused homologous or heterologous nucleic acid sequences, introduced into the cells or genome of a test subject. In the current invention, transgenes are carried on any viral vector that is used to introduce the transgenes to the cells of the subject.

The term “Second Generation Adenovirus”, as used herein, refers to an Ad that has all or parts of the E1, E2, E3, and, in certain embodiments, E4 DNA gene sequences deleted (removed) from the virus.

The term “subject”, as used herein, refers to any animal, e.g., a mammal or marsupial. Subjects include but are not limited to humans, non-human primates (e.g., rhesus or other types of macaques), mice, pigs, horses, donkeys, cows, sheep, rats and fowl of any kind.

In certain aspects, there may be provided methods for producing a vaccine that generates immune responses against various Influenza viruses using an adenovirus vector that allows for multiple vaccinations to generate broadly reactive immune responses against influenza viruses.

One aspect provides a method of generating an immune response against several influenza target antigens in an individual comprising administering to the individual an adenovirus vector comprising: a) a replication defective adenovirus vector, wherein the adenovirus vector has a deletion in the E2b region, and b) nucleic acids encoding multiple influenza target antigens; and readministering the adenovirus vector at least once to the individual; thereby generating an immune response against the influenza target antigens.

Another aspect provides a method for generating an immune response against several influenza target antigens in an individual, wherein the individual has preexisting immunity to adenovirus, comprising: administering to the individual an adenovirus vector comprising: a) a replication defective adenovirus vector, wherein the adenovirus vector has a deletion in the E2b region, and b) nucleic acids encoding multiple influenza target antigens; and readministering the adenovirus vector at least once to the individual; thereby generating an immune response against the influenza target antigens.

In a further aspect, the target antigens are comprised of antigens derived from influenza A and B virus proteins. In this regard, the influenza proteins may be derived from any influenza A and B viruses, including but not limited to H3N2, H9N1, H1N1, H2N2, H7N7, H1N2, H9N2, H7N2, H7N3, H10N7, influenza B/Yamagata, and influenza B/Victoria. In certain embodiments, the influenza virus protein may be any influenza protein, including but not limited to BM2 protein, hemagglutinin, hemagglutinin stalk, neuraminidase, nucleoprotein, matrix protein M1, and matrix protein M2.

### Need for Development of a Universal Influenza Vaccine and Importance of Immune Responses.

Pandemic influenza outbreaks are a major threat to global public health. Such outbreaks present the potential for sudden emergence and explosive transmission of virus strains to which humans have little or no immunity. Many of these virus strains cause severe or life-threatening illness requiring hospitalization. The most efficient way to prevent severe influenza is vaccination of the susceptible population. Conventional influenza vaccines function by inducing antibodies (Abs) against the highly variable surface glycoprotein hemagglutinin (HA), and mostly act by reducing viral infectivity and spreading in the infected individual. This type of vaccine currently takes at least 6-12 months to prepare and distribute once a potential pandemic strain has been identified, which is much too long. This was highlighted by the 2009 H1N1 pandemic, when the newly emergent virus was identified in April 2010 but sufficient vaccine for mass immunization was not available until October. Meanwhile the virus spread and the need for a vaccine was evident. This demonstrates the need for influenza vaccines that can be produced rapidly, especially for use in high-risk populations requiring a vaccine that induces the protective effects of both cellular and humoral immune responses against influenza.

### Influenza Antigens

In certain embodiments, influenza antigens such as hemagglutinin, nucleoprotein, and matrix components may be used, for example, in a vaccine composition or a composition comprising an adenoviral vector.

For example, hemagglutinin antigens may be used. The main correlate of protection against natural influenza infection is the level of Abs that are specific for HA in the serum and mucosa. Seasonal influenza vaccines are approved based on the induction of humoral responses to HA as measured by hemagglutination inhibition (HAI) assays. The HA antigen appears to contain conserved antigen epitopes in the stem region that are cross-reactive with influenza subtypes (Nabel G J Trans Am Clin Climatol Assoc. 2012;123:9-15).

M2 protein and nucleoprotein (NP) may also be used in certain aspects. Studies have shown that the influenza M2 protein and nucleoprotein (NP) also contain conserved regions that provide a wide range of influenza subtype-independent protection when used in experimental vaccines, including those employing Ad5 vectors (Epstein S L et al. Vaccine. 2005 23:5404-10; Tompkins S M et al. Emerg Infect Dis. 2007 13:426-35; Price G E et al. PLoS One. 2010 5(10):e13162; Osterhaus A Philos Trans R Soc Lond B Biol Sci. 2011 366(1579):2766-73).

Vaccination strategies have used the NP as an antigen to induce immune responses, since it is well conserved across influenza virus subtypes (Altstein A D et al. Arch Virol. 2006 151:921-31; Saha S et al. Virology. 2006 354:48-57; Goodman A G et al. PloS One. 2011 6(10):e25938).

In addition, the highly conserved membrane external domain within the M2 protein (M2e) has been studied as an attractive target for influenza A vaccine development (Tompkins S M et al. Emerg Infect Dis. 2007 13:426-35; Neirynck S et al. Nat Med. 1999 5:1157-63; Roose K et al. Drug News Perspect. 2009 22:80-92; Turley C B et al. Vaccine. 2011 29:5145-52). Humoral responses to M2e can inhibit influenza infection by mechanism(s) potentially involving Ab-dependent cell-mediated cytotoxicity and/or triggering the complement cascade, resulting in cytolysis (El Bakkouri K et al. J Immunol. 2011 186:1022-31). One research group (Zhou D et al. Mol Ther. 2010 18:2182-9) generated E1-deleted adenovirus (Ad) vectors from chimpanzee serotypes C68 (AdC68) or C6 (AdC6) that expressed, in tandem, three M2e sequences from diverse strains of influenza A virus (H1N1, H5N1, and H7N2) fused to H1N1 NP. The Ad vaccines expressing M2e and NP elicited robust NP-specific CD8⁺ T-cell responses and moderate antibody responses to the three M2e sequences in mice. Interestingly, vaccinated young mice are protected against mortality following challenge with high doses of different influenza viruses. The influenza B virus mutates slowly and its BM2 protein contains a highly conserved region among influenza strain B types that is an ideal candidate for a broad-based vaccine (Hiebert S W., Williams M A, Lamb R A Virology. 1986 155:747-51). The ability to induce both humoral and CMI responses against conserved influenza components such as BM2, M2, NP, and a consensus HA that results in protection against heterologous influenza viruses holds tremendous potential in the development of a broadly reactive influenza vaccine.

In developing a new universal influenza vaccine, all these above aspects are capitalized upon to develop a multi-strain cross-reactive influenza vaccine utilizing highly conserved and cross-reactive antigens of influenza type A and B.

### Adenoviral Vectors

In certain aspects, adenoviral vectors may be used in compositions and methods for the delivery of influenza antigens.

The recombinant Ad5 [E1-, E2b-] vector vaccine platform is new, having additional deletions in the early gene 2b (E2b) region that remove the viral DNA polymerase (pol) and/or the pre terminal protein (pTP) genes, and is propagated in the E.C7 human cell line (Amalfitano A, Begy C R, Chamberlain J S Proc Natl Acad Sci U S A. 1996 93:3352-6; Amalfitano A, Chamberlain J S Gene Ther. 1997 4:258-63; Amalfitano A et al. J Virol. 1998 72:926-33; Seregin S S and Amalfitano A Expert Opin Biol Ther. 2009 9:1521-31). The vector has an expanded gene-carrying/cloning capacity of up to 12 kb, compared to the 7 kb capacity of current Ad5 [E1-] vectors, which is sufficient to allow inclusion of multiple genes (Amalfitano A et al. J Virol. 1998 72:926-33; Seregin SS and Amalfitano A Expert Opin Biol Ther. 2009 9:1521-31). Additional deletions of the E2b region confers advantageous immune properties such as eliciting potent immune responses to specific antigens while minimizing immune responses to Ad5 viral proteins.

Importantly, pre-clinical and clinical studies in cancer and infectious disease demonstrate that Ad5 [E1-, E2b-]-based vectors induce potent CMI and Ab responses against vectored antigens, even in the presence of Ad5 immunity (Osada T et al. Cancer Gene Ther. 2009 16:673-82; Gabitzsch ES et al. Vaccine. 2009 27:6394-8; Gabitzsch E S et al. Immunol Lett. 2009 122:44-51; Gabitzsch ES et al. Cancer Immunol Immunother. 2010 59:1131-5; Gabitzsch E S et al. Cancer Gene Ther. 2011 18:326-35; Gabitzsch E S et al. Vaccine 2011 29:8101-7; Jones F R et al. Vaccine 2011 29:7020-6; Gabitzsch E S, Jones F R J Clin Cell Immunol. 2011 S4:001. doi:10.4172/2155-9899. S4-001; Gabitzsch E S et al. Vaccine 2012 30:7265-70; Wieking B G et al. Cancer Gene Ther. 2012 19:667-74; Morse M A et al. Cancer Immunol Immunother. 2013 62:1293-1301; Balint et al. Cancer Immunol Immunother. 2015 64:977-87; Rice A E et al. Cancer Gene Ther. 2015 22:454-62; Gabitzsch E S et al. Oncotarget 2015 Sep. 7 epub ahead of print).

The advanced recombinant adenovirus serotype 5 (Ad5) vector platform gives the opportunity to develop a novel broadly cross-reactive vaccine for influenza. This vector can be delivered directly by subcutaneous injection for exposure of defined influenza antigens to antigen-presenting cells (APCs) that induce potent immune responses. Importantly, the Ad5 recombinant vector replicates episomally and does not insert the genome into the host cell genome, thereby ensuring that there is no gene integration and disruption of vital cellular gene functions (Imler J L Vaccine. 1995 13:1143-51; Ertl H C, Xiang Z J Immunol. 1996 156:3579-82; Amalfitano, A Curr Opin Mol Ther. 2003 5:362-6).

Unfortunately, a major challenge facing current Ad5-based vectors is the presence of pre-existing immunity to Ad5. Most people exhibit neutralizing Abs against Ad5, the most widely used subtype for human vaccines, with two-thirds of people studied having lympho-proliferative responses against Ad5 (Chirmule N et al. Gene Ther. 1999 6:1574-83). This immunity prevents the use of current early gene 1 (E1) region-deleted Ad5 vectors (Ad5 [E1-]) as a platform for an influenza vaccine. Ad5 immunity inhibits immunization, and especially re-immunization with recombinant Ad5 vectors, and precludes immunization of a vaccine against a second disease antigen as well. Overcoming the problem of pre-existing Ad5 vector immunity has been the subject of intense investigation. However, use of other Ad serotypes or even non-human forms of Ad can lead directly to altered production of important chemokines and cytokines, gene dysregulation, and have significantly different biodistribution and tissue toxicities (Appledorn D M et al. Gene Ther. 2008 15:885-901; Hartman Z C et al. Virus Res. 2008 132:1-14). Even if these approaches succeed in an initial immunization, subsequent vaccinations are problematic due to induced immune responses to the Ad subtype. To help avoid the Ad immunization barrier and circumvent the adverse conditions for current Ad5 [E1-] vectors, an improved Ad5 vector platform was constructed, described above.

Further, the Ad5 [E1-, E2b-] vectors display reduced inflammation during the first 24 to 72 hours after injection compared to current Ad5 [E1-] vectors (Nazir S A, Metcalf J P J Investig Med. 2005 53:292-304; Schaack J Proc Natl Acad Sci U S A. 2004 101:3124-9; Schaack J Viral Immunol. 2005 18:79-88). The lack of Ad5 [E1-, E2b-] late gene expression renders infected cells less vulnerable to anti-Ad5 activity and permits them to produce and express the transgene for extended periods of time (Gabitzsch E S, Jones F R J Clin Cell Immunol. 2011 S4:001. doi:10.4172/2155-9899. S4-001; Hodges B L J Gene Med. 2000 2:250-9). Reduced inflammatory responses against Ad5 [E1-, E2b-] viral proteins and the resulting evasion of pre-existing Ad5 immunity may increases the ability of Ad5 [E1-, E2b-] to infect APC cells, resulting in greater immunization of the inoculee. In addition, increased infection of other cell types may provide the high levels of antigen presentation needed for potent CD4⁺ and CD8⁺ T cell responses, leading to memory T cell development. Thus it appears that deletion of the E2b region confers advantageous immune properties, such as eliciting potent immune responses to specific antigens, while minimizing immune responses to Ad5 proteins even in the presence of pre-existing Ad5 immunity.

It was reported in animal models of cancer (colorectal, breast, and HPV) (Osada T et al. Cancer Gene Ther. 2009 16:673-82; Gabitzsch E S et al. Cancer Immunol Immunother. 2010 59:1131-5; Gabitzsch ES et al. Cancer Gene Ther. 2011 18:326-35; Wieking BG et al. Cancer Gene Ther. 2012 19:667-74; Rice A E et al. Cancer Gene Ther. 2015 22:454-62; Gabitzsch E S et al. Oncotarget 2015 Sep. 7 epub ahead of print) and infectious disease (HIV, SIV, and H1N1 influenza) (Gabitzsch E S et al. Vaccine. 2009 27:6394-8; Gabitzsch E S et al. Vaccine 2011 29:8101-7; Jones F R et al. Vaccine 2011 29:7020-6; Gabitzsch E S et al. Vaccine 2012 30:7265-70) that potent immune responses are induced against expressed antigen genes even in the presence of Ad5 hyperimmunity. Of particular relevance are the studies with the new Ad5 [E1-, E2b-]-CEA (carcinoembryonic antigen) platform vaccine for immunotherapy in advanced stage colorectal cancer patents. CEA-directed CMI responses were induced despite pre-existing Ad5 immunity; treatments were well tolerated, safely administered, and no serious adverse effects were observed (Morse M A et al. Cancer Immunol Immunother. 2013 62:1293-1301; Balint et al. Cancer Immunol Immunother. 2015 64:977-87).

The results demonstrated the ability of recombinant Ad5 [E1-, E2b-] platform-based vaccines to overcome pre-existing and/or Ad5 vector-induced immunity and induce significant protective immune responses. These studies established that new Ad5 [E1-, E2b-] vector-based vaccines 1) can induce significantly higher CMI responses compared to current Ad5 [E1-] vectors, 2) can be utilized for multiple immunization regimens designed to induce potent CMI responses, 3) can induce significant antigen-specific CMI responses in animals with pre-existing Ad5 immunity, and 4) can induce significant anti-tumor responses or protect against infectious disease in animals with high levels of pre-existing Ad5 immunity.

In certain embodiments, the innovative attributes of the new Ad5 [E1-, E2b-] recombinant platform can be used to develop a broadly cross-reactive influenza vaccine. This may be accomplished by incorporating into the recombinant Ad5 [E1-, E2b-] platform multiple transgenes that express conserved and cross-reactive antigens from the HA, BM2, M2 and NP proteins of influenza A and B strains and to be utilized as a new universal influenza vaccine.

Certain aspects relate to methods and adenovirus vectors for generating immune responses against influenza target antigens. In particular, certain aspects may provide an improved Ad-based vaccine such that multiple vaccinations against more than one antigenic target entity can be achieved. Importantly, vaccination can be performed in the presence of preexisting immunity to the Ad and/or administered to subjects previously immunized multiple times with the adenovirus vector as described herein or other adenovirus vectors. The adenovirus vector can be administered to subjects multiple times to induce an immune response against a variety of influenza A and B antigens, including but not limited to, the production of broad based antibody and cell-mediated immune responses against influenza A and B viruses.

Certain aspects provide the use of E2b deleted adenovirus vectors, such as those described in U.S. Pat. Nos. 6,063,622; 6,451 ,596; 6,057,158: and 6,083,750 (all incorporated herein in their entirety by reference). As described in the '622 patent, in order to further cripple viral protein expression, and also to decrease the frequency of generating replication competent Ad (RCA), adenovirus vectors containing deletions in the E2b region may be provided in certain aspects. Propagation of these E2b deleted adenovirus vectors requires cell lines that express the deleted E2b gene products.

In further aspects, there may be provided packaging cell lines; for example E.C7 (formally called C-7), derived from the HEK-203 cell line (Amalfitano A et al. Proc Natl Acad Sci USA 1996 93:3352-56; Amalfitano A et al. Gene Ther 1997 4:258-63).

Further, the E2b gene products, DNA polymerase and preterminal protein, can be constitutively expressed in E.C7, or similar cells along with the El gene products. Transfer of gene segments from the Ad genome to the production cell line has immediate benefits: (1) increased carrying capacity of the recombinant DNA polymerase and preterminal protein-deleted adenovirus vector, since the combined coding sequences of the DNA polymerase and preterminal proteins that can be theoretically deleted approaches 4.6 kb; and, (2) a decreased potential of RCA generation, since two or more independent recombination events would be required to generate RCA.

Therefore, the E1, Ad DNA polymerase and preterminal protein expressing cell lines can enable the propagation of adenovirus vectors with a carrying capacity approaching 13 kb, without the need for a contaminating helper virus (Mitani et al. Proc. Natl. Acad. Sci. USA 1995 92:3854; Hodges et al. J Gene Med 2000 2:250-259; Amalfitano and Parks Curr Gene Ther 2002 2:111-133).

In addition, when genes critical to the viral life cycle are deleted (e.g., the E2b genes), a further crippling of Ad to replicate or express other viral gene proteins occurs. This will decrease immune recognition of virally infected cells, and allows for extended durations of foreign transgene expression.

Important attributes of E1, DNA polymerase, and preterminal protein deleted vectors, however, include their inability to express the respective proteins from the E1 and E2b regions, as well as a predicted lack of expression of most of the viral structural proteins. For example, the major late promoter (MLP) of Ad is responsible for transcription of the late structural proteins L1 through L5 (Doerfler, In Adenovirus DNA, The Viral Genome and Its Expression (Martinus Nijhoff Publishing Boston, 1986). Though the MLP is minimally active prior to Ad genome replication, the highly toxic Ad late genes are primarily transcribed and translated from the MLP only after viral genome replication has occurred (Thomas and Mathews Cell 1980 22:523). This cis-dependent activation of late gene transcription is a feature of DNA viruses in general, such as in the growth of polyoma and SV-40. The DNA polymerase and preterminal proteins are absolutely required for Ad replication (unlike the E4 or protein IX proteins) and thus their deletion is extremely detrimental to adenovirus vector late gene expression, and the toxic effects of that expression in cells such as APCs.

In certain embodiments, the adenovirus vectors contemplated for use include E2b deleted adenovirus vectors that have a deletion in the E2b region of the Ad genome and the E1 region but do not have any other regions of the Ad genome deleted. In another embodiment, the adenovirus vectors contemplated for use may include E2b deleted adenovirus vectors that have a deletion in the E2b region of the Ad genome and deletions in the E1 and E3 regions, but no other regions deleted. In a further embodiment, the adenovirus vectors contemplated for use may include adenovirus vectors that have a deletion in the E2b region of the Ad genome and deletions in the E1, E3 and partial or complete removal of the E4 regions but no other deletions.

In another embodiment, the adenovirus vectors contemplated for use include adenovirus vectors that have a deletion in the E2b region of the Ad genome and deletions in the E1 and E4 regions but no other deletions. In an additional embodiment, the adenovirus vectors contemplated for use may include adenovirus vectors that have a deletion in the E2a, E2b and E4 regions of the Ad genome but no other deletions.

In one embodiment, the adenovirus vectors for use herein comprise vectors having the E1 and DNA polymerase functions of the E2b region deleted but no other deletions. In a further embodiment, the adenovirus vectors for use herein have the El and the preterminal protein functions of the E2b region deleted and no other deletions.

In another embodiment, the adenovirus vectors for use herein have the E1, DNA polymerase and the preterminal protein functions deleted, and no other deletions. In one particular embodiment, the adenovirus vectors contemplated for use herein are deleted for at least a portion of the E2b region and the E1 region, but are not “gutted” adenovirus vectors. In this regard, the vectors may be deleted for both the DNA polymerase and the preterminal protein functions of the E2b region.

The term “E2b deleted”, as used herein, refers to a specific DNA sequence that is mutated in such a way so as to prevent expression and/or function of at least one E2b gene product. Thus, in certain embodiments, “E2b deleted” refers to a specific DNA sequence that is deleted (removed) from the Ad genome. E2b deleted or “containing a deletion within the E2b region” refers to a deletion of at least one base pair within the E2b region of the Ad genome. Thus, in certain embodiments, more than one base pair is deleted and in further embodiments, at least 20, 30, 40, 50, 60, 70, 80, 90, 100, 110, 120, 130, 140, or 150 base pairs are deleted. In another embodiment, the deletion is of more than 150, 160, 170, 180, 190, 200, 250, or 300 base pairs within the E2b region of the Ad genome. An E2b deletion may be a deletion that prevents expression and/or function of at least one E2b gene product and therefore, encompasses deletions within exons encoding portions of E2b-specific proteins as well as deletions within promoter and leader sequences. In certain embodiments, an E2b deletion is a deletion that prevents expression and/or function of one or both of the DNA polymerase and the preterminal protein of the E2b region. In a further embodiment, “E2b deleted” refers to one or more point mutations in the DNA sequence of this region of an Ad genome such that one or more encoded proteins is non-functional. Such mutations include residues that are replaced with a different residue leading to a change in the amino acid sequence that result in a nonfunctional protein.

As would be understood by the skilled artisan upon reading the present disclosure, other regions of the Ad genome can be deleted. Thus to be “deleted” in a particular region of the Ad genome, as used herein, refers to a specific DNA sequence that is mutated in such a way so as to prevent expression and/or function of at least one gene product encoded by that region. In certain embodiments, to be “deleted” in a particular region refers to a specific DNA sequence that is deleted (removed) from the Ad genome in such a way so as to prevent the expression and/or the function encoded by that region (e.g., E2b functions of DNA polymerase or preterminal protein function). “Deleted” or “containing a deletion” within a particular region refers to a deletion of at least one base pair within that region of the Ad genome. Thus, in certain embodiments, more than one base pair is deleted and in further embodiments, at least 20, 30, 40, 50, 60, 70, 80, 90, 100, 110, 120, 130, 140, or 150 base pairs are deleted from a particular region. In another embodiment, the deletion is more than 150, 160, 170, 180, 190, 200, 250, or 300 base pairs within a particular region of the Ad genome.

These deletions are such that expression and/or function of the gene product encoded by the region may be prevented. Thus deletions encompass deletions within exons encoding portions of proteins as well as deletions within promoter and leader sequences. In a further embodiment, “deleted” in a particular region of the Ad genome refers to one or more point mutations in the DNA sequence of this region of an Ad genome such that one or more encoded proteins is non-functional. Such mutations include residues that are replaced with a different residue leading to a change in the amino acid sequence that result in a nonfunctional protein.

The adenovirus vectors comprising one or more deletions can be generated using recombinant techniques known in the art (see e.g., Amalfitano et al. J. Virol. 1998 72:926-33; Hodges, et al., J Gene Med 2000 2:250-59). As would be recognized by the skilled artisan, the adenovirus vectors for use can be successfully grown to high titers using an appropriate packaging cell line that constitutively expresses E2b gene products and products of any of the necessary genes that may have been deleted. In certain embodiments, HEK-293-derived cells that not only constitutively express the E1 and DNA polymerase proteins, but also the Ad-preterminal protein, can be used. In one embodiment, E.C7 cells are used to successfully grow high titer stocks of the adenovirus vectors (see e.g., Amalfitano et al. J. Virol. 1998 72:926-33; Hodges et al. J Gene Med 2000 2:250-59).

In order to delete critical genes from self-propagating adenovirus vectors, the proteins encoded by the targeted genes have to first be coexpressed in HEK-293 cells, or similar, along with the E1 proteins. Therefore, only those proteins which are non-toxic when coexpressed constitutively (or toxic proteins inducibly-expressed) can be utilized. Coexpression in HEK-293 cells of the El and E4 genes has been demonstrated (utilizing inducible, not constitutive, promoters) (Yeh et al. J. Virol. 1996 70:559; Wang et al. Gene Therapy 1995 2:775; and Gorziglia et al. J. Virol. 1996 70:4173). The E1 and protein IX genes (a virion structural protein) have been coexpressed (Caravokyri and Leppard J. Virol. 1995 69:6627), and coexpression of the E1, E4, and protein IX genes has also been described (Krougliak and Graham Hum. Gene Ther. 1995 6:1575). The E1 and 100 k genes have been successfully expressed in transcomplementing cell lines, as have El and protease genes (Oualikene et al. Hum Gene Ther 2000 11 :1341-53; Hodges et al. J. Virol 2001 75:5913-20).

Cell lines coexpressing E1 and E2b gene products for use in growing high titers of E2b deleted Ad particles are described in U.S. Pat. No. 6,063,622. The E2b region encodes the viral replication proteins which are absolutely required for Ad genome replication (Doerfler, supra and Pronk et al. Chromosoma 1992 102:S39-S45). Useful cell lines constitutively express the approximately 140 kDa Ad-DNA polymerase and/or the approximately 90 kDa preterminal protein. In particular, cell lines that have high-level, constitutive coexpression of the E1, DNA polymerase, and preterminal proteins, without toxicity (e.g., E.C7), are desirable for use in propagating Ad for use in multiple vaccinations. These cell lines permit the propagation of adenovirus vectors deleted for the E1, DNA polymerase, and preterminal proteins.

The recombinant Ad can be propagated using techniques known in the art. For example, in certain embodiments, tissue culture plates containing E.C7 cells are infected with the adenovirus vector virus stocks at an appropriate MOI (e.g., 5) and incubated at 37.0° C. for 40-96 h. The infected cells are harvested, resuspended in 10 mM Tris-Cl (pH 8.0), and sonicated, and the virus is purified by two rounds of cesium chloride density centrifugation. In certain techniques, the virus containing band is desalted over a Sephadex CL-6B column (Pharmacia Biotech, Piscataway, N.J.), sucrose or glycerol is added, and aliquots are stored at −80 ° C. In some embodiments, the virus will be placed in a solution designed to enhance its stability, such as A195 (Evans et al. J Pharm Sci 2004 93:2458-75). The titer of the stock is measured (e.g., by measurement of the optical density at 260 nm of an aliquot of the virus after SDS lysis). In another embodiment, plasmid DNA, either linear or circular, encompassing the entire recombinant E2b deleted adenovirus vector can be transfected into E.C7, or similar cells, and incubated at 37.0° C. until evidence of viral production is present (e.g., the cytopathic effect). The conditioned media from these cells can then be used to infect more E.C7, or similar cells, to expand the amount of virus produced, before purification.

Purification can be accomplished by two rounds of cesium chloride density centrifugation or selective filtration. In certain embodiments, the virus may be purified by column chromatography, using commercially available products (e.g., Adenopure from Puresyn, Inc., Malvem, Pa.) or custom made chromatographic columns.

In certain embodiments, the recombinant Ad may comprise enough of the virus to ensure that the cells to be infected are confronted with a certain number of viruses. Thus, there may be provided a stock of recombinant Ad, particularly, an RCA-free stock of recombinant Ad. The preparation and analysis of Ad stocks is well known in the art. Viral stocks vary considerably in titer, depending largely on viral genotype and the protocol and cell lines used to prepare them. The viral stocks can have a titer of at least about 10⁶, 10⁷, or 10⁶ pfu/ml, and many such stocks can have higher titers, such as at least about 10⁹, 10¹⁰, 10¹¹, or 10¹² pfu/ml.

### Heterologous Nucleic Acids

The adenovirus vectors also comprise heterologous nucleic acid sequences that encode several target antigens of interest, fragments or fusions thereof, against which it is desired to generate an immune response. In some embodiments, the adenovirus vectors comprise heterologous nucleic acid sequences that encode several proteins, fusions thereof or fragments thereof, which can modulate the immune response. Thus, certain aspects provide the Second Generation E2b deleted adenovirus vectors that comprise a heterologous nucleic acid sequence.

As such, certain aspects provide nucleic acid sequences, also referred to herein as polynucleotides that encode several influenza target antigens of interest. As such, certain aspects provide polynucleotides that encode target antigens from any source as described further herein, vectors comprising such polynucleotides and host cells transformed or transfected with such expression vectors. The terms “nucleic acid” and “polynucleotide” are used essentially interchangeably herein. As will be also recognized by the skilled artisan, polynucleotides may be single-stranded (coding or antisense) or double-stranded, and may be DNA (genomic, cDNA or synthetic) or RNA molecules. RNA molecules may include HnRNA molecules, which contain introns and correspond to a DNA molecule in a one-to-one manner, and mRNA molecules, which do not contain introns. Additional coding or non-coding sequences may, but need not, be present within a polynucleotide, and a polynucleotide may, but need not, be linked to other molecules and/or support materials. An isolated polynucleotide, as used herein, means that a polynucleotide is substantially away from other coding sequences. For example, an isolated DNA molecule as used herein does not contain large portions of unrelated coding DNA, such as large chromosomal fragments or other functional genes or polypeptide coding regions. Of course, this refers to the DNA molecule as originally isolated, and does not exclude genes or coding regions later added to the segment recombinantly in the laboratory.

As will be understood by those skilled in the art, the polynucleotides can include genomic sequences, extra-genomic and plasmid-encoded sequences and smaller engineered gene segments that express, or may be adapted to express target antigens as described herein, fragments of antigens, peptides and the like. Such segments may be naturally isolated, or modified synthetically by the hand of man.

Polynucleotides may comprise a native sequence (i.e., an endogenous sequence that encodes a target antigen polypeptide/protein/epitope or a portion thereof) or may comprise a sequence that encodes a variant or derivative of such a sequence. In certain embodiments, the polynucleotide sequences set forth herein encode target antigen proteins as described herein. In some embodiments, polynucleotides represent a novel gene sequence that has been optimized for expression in specific cell types (i.e., human cell lines) that may substantially vary from the native nucleotide sequence or variant but encode a similar protein antigen.

In other related embodiments, there may be provided polynucleotide variants having substantial identity to native sequences encoding proteins (e.g., target antigens of interest) as described herein, for example those comprising at least 70% sequence identity, particularly at least 75% up to 99% or higher, sequence identity compared to a native polynucleotide sequence encoding the polypeptides using the methods described herein, (e.g., BLAST analysis using standard parameters, as described below). One skilled in this art will recognize that these values can be appropriately adjusted to determine corresponding identity of proteins encoded by two nucleotide sequences by taking into account codon degeneracy, amino acid similarity, reading frame positioning and the like.

In certain aspects, polynucleotide variants will contain one or more substitutions, additions, deletions and/or insertions, particularly such that the immunogenicity of the epitope of the polypeptide encoded by the variant polynucleotide or such that the immunogenicity of the heterologous target protein is not substantially diminished relative to a polypeptide encoded by the native polynucleotide sequence. As described elsewhere herein, the polynucleotide variants may encode a variant of the target antigen, or a fragment (e.g., an epitope) thereof wherein the propensity of the variant polypeptide or fragment (e.g., epitope) thereof to react with antigen-specific antisera and/or T-cell lines or clones is not substantially diminished relative to the native polypeptide. The term “variants” should also be understood to encompass homologous genes of xenogeneic origin.

Certain aspects may provide polynucleotides that comprise or consist of at least about 5 up to a 1000 or more contiguous nucleotides encoding a polypeptide, including target protein antigens, as described herein, as well as all intermediate lengths there between. It will be readily understood that “intermediate lengths”, in this context, means any length between the quoted values, such as 16, 17, 18, 19, etc.; 21, 22, 23, etc.; 30, 31, 32, etc.; 50, 51, 52, 53, etc.; 100, 101, 102, 103, etc.; 150, 151, 152, 153, etc.; including all integers through 200-500; 500-1,000, and the like. A polynucleotide sequence as described herein may be extended at one or both ends by additional nucleotides not found in the native sequence encoding a polypeptide as described herein, such as an epitope or heterologous target protein. This additional sequence may consist of 1 up 20 nucleotides or more, at either end of the disclosed sequence or at both ends of the disclosed sequence.

In certain embodiments, the polynucleotides, or fragments thereof, regardless of the length of the coding sequence itself, may be combined with other DNA sequences, such as promoters, expression control sequences, polyadenylation signals, additional restriction enzyme sites, multiple cloning sites, other coding segments, and the like, such that their overall length may vary considerably. It is therefore contemplated that a nucleic acid fragment of almost any length may be employed, with the total length that may limited by the ease of preparation and use in the intended recombinant DNA protocol. For example, illustrative polynucleotide segments with total lengths of about 1000, 2000, 3000, 4000, 5000, 6000, 7000, 8000, 9000, 10,000, about 500, about 200, about 100, about 50 base pairs in length, and the like, (including all intermediate lengths) are contemplated to be useful in many implementations.

When comparing polynucleotide sequences, two sequences are said to be “identical” if the sequence of nucleotides in the two sequences is the same when aligned for maximum correspondence, as described below. Comparisons between two sequences may be performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff MO (1978) A model of evolutionary change in proteins—Matrices for detecting distant relationships. In Dayhoff MO (ed.) Atlas of Protein Sequence and Structure, National Biomedical Research Foundation, Washington D.C. Vol. 5, Suppl. 3, pp. 345-358; Hein J Unified Approach to Alignment and Phylogenes, pp. 626-645 (1990); Methods in Enzymology vol.183, Academic Press, Inc., San Diego, Calif.; Higgins D G and Sharp P M CABIOS 1989 5:151-53; Myers E W and Muller W CABIOS 1988 4:11-17; Robinson E D Comb. Theor 1971 11A 05; Saitou N, Nei M Mol. Biol. Evol. 1987 4:406-25; Sneath PHA and Sokal RR Numerical Taxonomy—the Principles and Practice of Numerical Taxonomy, Freeman Press, San Francisco, Calif. (1973); Wilbur W J and Lipman D J Proc. Natl. Acad., Sci. USA 1983 80:726-30).

Alternatively, optimal alignment of sequences for comparison may be conducted by the local identity algorithm of Smith and Waterman, Add. APL. Math 1981 2:482, by the identity alignment algorithm of Needleman and Wunsch J. Mol. Biol. 1970 48:443, by the search for similarity methods of Pearson and Lipman, Proc. Natl. Acad. Sci. USA 1988 85:2444, by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

One example of algorithms that are suitable for determining percent sequence identity and sequence similarity are the BLAST and BLAST 2.0 algorithms, which are described in Altschul et al., Nucl. Acids Res. 1977 25:3389-3402, and Altschul et al. J. Mol. Biol. 1990 215:403-10, respectively. BLAST and BLAST 2.0 can be used, for example with the parameters described herein, to determine percent sequence identity for the polynucleotides. Software for performing BLAST analyses is publicly available through the National Center for Biotechnology Information. In one illustrative example, cumulative scores can be calculated using, for nucleotide sequences, the parameters M (reward score for a pair of matching residues; always >0) and N (penalty score for mismatching residues; always <0). Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLASTN program (for nucleotide sequences) uses as defaults a word length (W) of 11, and expectation (E) of 10, and the BLOSUM62 scoring matrix (see Henikoff and Henikoff, Proc. Natl. Acad. Sci. USA 1989 89:10915) alignments, (B) of 50, expectation (E) of 10, M=5, N=−4 and a comparison of both strands.

In certain aspects, the “percentage of sequence identity” is determined by comparing two optimally aligned sequences over a window of comparison of at least 20 positions, wherein the portion of the polynucleotide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases occurs in both sequences to yield the number of matched positions, dividing the number of matched positions by the total number of positions in the reference sequence (i.e., the window size) and multiplying the results by 100 to yield the percentage of sequence identity.

It will be appreciated by those of ordinary skill in the art that, as a result of the degeneracy of the genetic code, there are many nucleotide sequences that encode a particular antigen of interest, or fragment thereof, as described herein. Some of these polynucleotides bear minimal homology to the nucleotide sequence of any native gene. Nonetheless, polynucleotides that vary due to differences in codon usage are specifically contemplated in certain aspects. Further, alleles of the genes comprising the polynucleotide sequences provided herein are also contemplated. Alleles are endogenous genes that are altered as a result of one or more mutations, such as deletions, additions and/or substitutions of nucleotides. The resulting mRNA and protein may, but need not, have an altered structure or function. Alleles may be identified using standard techniques (such as hybridization, amplification and/or database sequence comparison).

Therefore, in another embodiment, a mutagenesis approach, such as site-specific mutagenesis, is employed for the preparation of variants and/or derivatives of the target antigen sequences, or fragments thereof, as described herein. By this approach, specific modifications in a polypeptide sequence can be made through mutagenesis of the underlying polynucleotides that encode them. These techniques provide a straightforward approach to prepare and test sequence variants, for example, incorporating one or more of the foregoing considerations, by introducing one or more nucleotide sequence changes into the polynucleotide.

Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Mutations may be employed in a selected polynucleotide sequence to improve, alter, decrease, modify, or otherwise change the properties of the polynucleotide itself, and/or alter the properties, activity, composition, stability, or primary sequence of the encoded polypeptide.

Polynucleotide segments or fragments encoding the polypeptides may be readily prepared by, for example, directly synthesizing the fragment by chemical means, as is commonly practiced using an automated oligonucleotide synthesizer. Also, fragments may be obtained by application of nucleic acid reproduction technology, such as the PCR™ technology of U.S. Pat. No. 4,683,202, by introducing selected sequences into recombinant vectors for recombinant production, and by other recombinant DNA techniques generally known to those of skill in the art of molecular biology (see for example, Current Protocols in Molecular Biology, John Wiley and Sons, NY, N.Y.).

In order to express a desired target antigen polypeptide or fragment thereof, or fusion protein comprising any of the above, as described herein, the nucleotide sequences encoding the polypeptide, or functional equivalents, are inserted into an appropriate Ad as described elsewhere herein using recombinant techniques known in the art. The appropriate adenovirus vector contains the necessary elements for the transcription and translation of the inserted coding sequence and any desired linkers. Methods that are well known to those skilled in the art may be used to construct these adenovirus vectors containing sequences encoding a polypeptide of interest and appropriate transcriptional and translational control elements. These methods include in vitro recombinant DNA techniques, synthetic techniques, and in vivo genetic recombination. Such techniques are described, for example, in Amalfitano et al. J. Virol. 1998 72:926-33; Hodges et al. J Gene Med 2000 2:250-259; Sambrook J et al. (1989) Molecular Cloning, A Laboratory Manual, Cold Spring Harbor Press, Plainview, N.Y., and Ausubel FM et al. (1989) Current Protocols in Molecular Biology, John Wiley & Sons, New York. N.Y.

A variety of vector/host systems may be utilized to contain and produce polynucleotide sequences. These include, but are not limited to, microorganisms such as bacteria transformed with recombinant bacteriophage, plasmid, or cosmid DNA vectors; yeast transformed with yeast vectors; insect cell systems infected with virus vectors (e.g., baculovirus); plant cell systems transformed with virus vectors (e.g., cauliflower mosaic virus, CaMV; tobacco mosaic virus, TMV) or with bacterial vectors (e.g., Ti or pBR322 plasmids); or animal cell systems.

The “control elements” or “regulatory sequences” present in an adenovirus vector are those non-translated regions of the vector—enhancers, promoters, 5′ and 3′ untranslated regions—which interact with host cellular proteins to carry out transcription and translation. Such elements may vary in their strength and specificity. Depending on the vector system and host utilized, any number of suitable transcription and translation elements, including constitutive and inducible promoters, may be used. For example, sequences encoding a polypeptide of interest may be ligated into an Ad transcription/translation complex consisting of the late promoter and tripartite leader sequence. Insertion in a non-essential E1 or E3 region of the viral genome may be used to obtain a viable virus that is capable of expressing the polypeptide in infected host cells (Logan J and Shenk T (1984) Proc. Natl. Acad. Sci 1984 87:3655-59). In addition, transcription enhancers, such as the Rous sarcoma virus (RSV) enhancer, may be used to increase expression in mammalian host cells.

Specific initiation signals may also be used to achieve more efficient translation of sequences encoding a polypeptide of interest. Such signals include the ATG initiation codon and adjacent sequences. In cases where sequences encoding the polypeptide, its initiation codon, and upstream sequences are inserted into the appropriate expression vector, no additional transcriptional or translational control signals may be needed. However, in cases where only coding sequence, or a portion thereof, is inserted, exogenous translational control signals including the ATG initiation codon should be provided. Furthermore, the initiation codon should be in the correct reading frame to ensure translation of the entire insert. Exogenous translational elements and initiation codons may be of various origins, both natural and synthetic. The efficiency of expression may be enhanced by the inclusion of enhancers that are appropriate for the particular cell system which is used, such as those described in the literature (Scharf D. et al. Results Probl. Cell Differ. 1994 20:125-62). Specific termination sequences, either for transcription or translation, may also be incorporated in order to achieve efficient translation of the sequence encoding the polypeptide of choice.

A variety of protocols for detecting and measuring the expression of polynucleotide-encoded products (e.g., target antigens of interest), using either polyclonal or monoclonal antibodies specific for the product are known in the art. Examples include enzyme-linked immunosorbent assay (ELISA), radioimmunoassay (RIA), and fluorescence activated cell sorting (FACS). A two-site, monoclonal-based immunoassay utilizing monoclonal antibodies reactive to two non-interfering epitopes on a given polypeptide may be used for some applications, but a competitive binding assay may also be employed. These and other assays are described, among other places, in Hampton R et al. (1990; Serological Methods, a Laboratory Manual, APS Press, St Paul. Minn.) and Maddox D E et al. J. Exp. Med. 1983 758:1211-16). The adenovirus vectors may comprise nucleic acid sequences encoding several influenza antigens of interest.

In certain embodiments, elements that increase the expression of the desired target antigen are incorporated into the nucleic acid sequence of the adenovirus vectors described herein. Such elements include internal ribosome binding sites (IRES; Wang and Siddiqui Curr. Top. Microbiol. Immunol 1995 203:99; Ehrenfeld and Semler Curr. Top. Microbiol. Immunol. 1995 203:65; Rees et al., Biotechniques 1996 20:102; Sugimoto et al. Biotechnology 1994 2:694). IRES increase translation efficiency. As well, other sequences may enhance expression. For some genes, sequences especially at the 5′ end inhibit transcription and/or translation. These sequences are usually palindromes that can form hairpin structures. Any such sequences in the nucleic acid to be delivered may be deleted or not deleted.

Expression levels of the transcript or translated product may be assayed to confirm or ascertain which sequences affect expression. Transcript levels may be assayed by any known method, including Northern blot hybridization, RNase probe protection and the like. Protein levels may be assayed by any known method, including ELISA. As would be recognized by the skilled artisan, the adenovirus vectors comprising heterologous nucleic acid sequences can be generated using recombinant techniques known in the art, such as those described in Maione et al. Proc Natl Acad Sci USA 2001 98:5986-91; Maione et al. Hum Gene Ther 2000 1:859-68; Sandig et al. Proc Natl Acad Sci USA, 2000 97:1002-07; Harui et al. Gene Therapy 2004 11:1617-26; Parks et al. Proc Natl Acad Sci USA 1996 93:13565-570; DelloRusso et al. Proc Natl Acad Sci USA 2002 99:12979-984; Current Protocols in Molecular Biology, John Wiley and Sons, NY, N.Y.).

As noted above, the adenovirus vectors comprise nucleic acid sequences that encode several influenza target proteins or antigens of interest. In this regard, the vectors may contain nucleic acid encoding 1 to 4 or more different target antigens of interest. The target antigens may be a full length protein or may be a fragment (e.g., an epitope) thereof. The adenovirus vectors may contain nucleic acid sequences encoding multiple fragments or epitopes from one target protein of interest or may contain one or more fragments or epitopes from numerous different target influenza antigen proteins of interest.

In certain embodiments, immunogenic fragments bind to an MHC class I or class Il molecule. As used herein, an immunogenic fragment is said to “bind to” an MHC class I or class Il molecule if such binding is detectable using any assay known in the art. For example, the ability of a polypeptide to bind to MHC class I may be evaluated indirectly by monitoring the ability to promote incorporation of ¹²⁵I labeled β2-microglobulin ((β2m) into MHC class I/β2m/peptide heterotrimeric complexes (see Parker et al., J. Immunol. 752:163, 1994). Alternatively, functional peptide competition assays that are known in the art may be employed. Immunogenic fragments of polypeptides may generally be identified using well known techniques, such as those summarized in Paul, Fundamental Immunology, 3rd ed., 243-247 (Raven Press, 1993) and references cited therein. Representative techniques for identifying immunogenic fragments include screening polypeptides for the ability to react with antigen-specific antisera and/or T-cell lines or clones. An immunogenic fragment of a particular target polypeptide is a fragment that reacts with such antisera and/or T-cells at a level that is not substantially less than the reactivity of the full length target polypeptide (e.g., in an ELISA and/or T-cell reactivity assay). In other words, an immunogenic fragment may react within such assays at a level that is similar to or greater than the reactivity of the full length polypeptide. Such screens may generally be performed using methods well known to those of ordinary skill in the art, such as those described in Harlow and Lane, Antibodies: A Laboratory Manual, Cold Spring Harbor Laboratory, 1988.

Target antigens include but are not limited to antigens derived from any of the influenza A and B viruses. Target antigens may include proteins produced by any of the infectious influenza viruses described herein, such as, but not limited to, viral antigen proteins, i.e., influenza BM2 protein, hemagglutinin, matrix protein M1, matrix protein M2, nucleoprotein, and neuraminidase. As used herein, an “infectious agent” is any living organism capable of infecting a host. Infectious agents include, for example, any variety of influenza A and B viruses.

The adenovirus vector may also include nucleic acid sequences that encode proteins that increase the immunogenicity of the target antigen. In this regard, the protein produced following immunization with the adenovirus vector containing such a protein may be a fusion protein comprising the target antigen of interest fused to a protein that increases the immunogenicity of the target antigen of interest.

### Methods of Use

The adenovirus vectors can be used in a number of vaccine settings for generating an immune response against one or more target antigens as described herein. The adenovirus vectors are of particular importance because of the unexpected finding that they can be used to generate immune responses in subjects who have preexisting immunity to Ad and can be used in vaccination regimens that include multiple rounds of immunization using the adenovirus vectors, regimens not possible using previous generation adenovirus vectors.

Generally, generating an immune response comprises an induction of a humoral response and/or a cell-mediated response. In certain embodiments, it is desirable to increase an immune response against a target antigen of interest. As such “generating an immune response” or “inducing an immune response” comprises any statistically significant change, e.g., increase in the number of one or more immune cells (T cells, B cells, antigen-presenting cells, dendritic cells, neutrophils, and the like) or in the activity of one or more of these immune cells (CTL activity, HTL activity, cytokine secretion, change in profile of cytokine secretion, etc.).

The skilled artisan would readily appreciate that a number of methods for establishing whether an alteration in the immune response has taken place are available. A variety of methods for detecting alterations in an immune response (e.g., cell numbers, cytokine expression, cell activity) are known in the art and are useful in the context of the instant invention. Illustrative methods are described in Current Protocols in Immunology, Edited by: John E. Coligan, Ada M. Kruisbeek, David H. Margulies, Ethan M. Shevach, Warren Strober (2001 John Wiley & Sons, NY, N.Y.) Ausubel et al. (2001 Current Protocols in Molecular Biology, Greene Publ. Assoc. Inc. & John Wiley & Sons, Inc., NY, N.Y.); Sambrook et al. (1989 Molecular Cloning, Second Ed., Cold Spring Harbor Laboratory, Plainview, N.Y.); Maniatis et al. (1982 Molecular Cloning, Cold Spring Harbor Laboratory, Plainview, N.Y.) and elsewhere. Illustrative methods useful in this context include intracellular cytokine staining (ICS), ELISpot, proliferation assays, cytotoxic T cell assays including chromium release or equivalent assays, and gene expression analysis using any number of polymerase chain reaction (PCR) or RT-PCR based assays.

In certain embodiments, generating an immune response comprises an increase in target antigen-specific CTL activity of about 1.5 to 20 or more fold, at least, about, or at most 1.5, 2, 3, 4, 5, 6, 7, 8, 9, 10, 11, 12, 13, 14, 15, 16, 17, 18, 19, or any range or number derived therefrom in a subject administered the adenovirus vectors as compared to a control. In another embodiment, generating an immune response comprises an increase in target-specific CTL activity of about 1.5 to 20, or more fold in a subject administered the adenovirus vectors as compared to a control. In a further embodiment, generating an immune response that comprises an increase in target antigen-specific cell mediated immunity activity as measured by ELISpot assays measuring cytokine secretion, such as interferon-gamma (IFN-γ), interleukin-2 (IL-2), tumor necrosis factor-alpha (TNF-α), granzyme, or other cytokines, of about 1.5 to 20, or more fold as compared to a control.

In a further embodiment, generating an immune response comprises an increase in target-specific antibody production of between 1.5 and 5 fold in a subject administered the adenovirus vectors as compared to an appropriate control. In another embodiment, generating an immune response comprises an increase in target-specific antibody production of about 1.5 to 20, or more fold in a subject administered the adenovirus vector as compared to a control.

Thus, certain aspects may provide methods for generating an immune response against influenza virus target antigens of interest comprising administering to the individual an adenovirus vector comprising: a) a replication defective adenovirus vector, wherein the adenovirus vector has a deletion in the E2b region, and b) nucleic acids encoding the target antigens; and readministering the adenovirus vector at least once to the individual; thereby generating an immune response against the target antigens. In certain embodiments, there may be provided methods wherein the vector administered is not a gutted vector.

In a further embodiment, methods may be provided for generating an immune response against influenza virus target antigens in an individual, wherein the individual has pre-existing immunity to Ad, by administering to the individual an adenovirus vector comprising: a) a replication defective adenovirus vector, wherein the adenovirus vector has a deletion in the E2b region, and b) nucleic acids encoding the target antigens; and re-administering the adenovirus vector at least once to the individual; thereby generating an immune response against the influenza virus target antigens.

With regard to preexisting immunity to Ad, this can be determined using methods known in the art, such as antibody-based assays to test for the presence of Ad antibodies. Further, in certain embodiments, the methods may include first determining that an individual has preexisting immunity to Ad then administering the E2b deleted adenovirus vectors as described herein.

In certain aspects, there may be provided methods of generating an immune response against influenza target antigens, such as those described elsewhere herein.

In particular aspects, there may be provided methods of generating an immune response against influenza A and B viruses, such as those described elsewhere herein.

As noted elsewhere herein, the adenovirus vectors comprise nucleic acid sequences that encode one or more target antigens of interest from any one or more of the infectious agents against which an immune response is to be generated. For example, target antigens may include, but are not limited to, viral antigen proteins, i.e., influenza BM2 protein, hemagglutinin, matrix protein M1, matrix protein M2, nucleoprotein, and neuraminidase.

For administration, the adenovirus vector stock may be combined with an appropriate buffer, physiologically acceptable carrier, excipient or the like. In certain embodiments, an appropriate number of adenovirus vector particles are administered in an appropriate buffer, such as, sterile PBS.

In certain circumstances it may be desirable to deliver the adenovirus vector compositions disclosed herein parenterally, intravenously, intramuscularly, or even intraperitoneally. In certain embodiments, solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. In other embodiments, E2b deleted adenovirus vectors may be delivered in pill form, delivered by swallowing or by suppository.

Illustrative pharmaceutical forms suitable for injectable use include sterile aqueous solutions or dispersions and sterile powders for the extemporaneous preparation of sterile injectable solutions or dispersions (for example, see U.S. Pat. No. 5,466,468). In all cases the form must be sterile and must be fluid to the extent that the formulation easily is pulled up and pushed through a syringe. It must be stable under the conditions of manufacture and storage and must be preserved against the contaminating action of microorganisms, such as bacteria, molds and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and/or by the use of surfactants. The prevention of the action of microorganisms can be facilitated by various antibacterial and antifungal agents, for example, parabens, chlorobutanol, phenol, sorbic acid, thimerosal, and the like. In many cases, it may include isotonic agents, for example, sugars or sodium chloride. Prolonged absorption of the injectable compositions can be brought about by the use in the compositions of agents delaying absorption, for example, aluminum monostearate and gelatin.

In one embodiment, for parenteral administration in an aqueous solution, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. Moreover, for human administration, preparations may need to meet sterility, pyrogenicity, and the general safety and purity standards as required by FDA Office of Biology standards.

The carriers can further comprise any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions. The phrase “pharmaceutically-acceptable” refers to molecular entities and compositions that do not produce an allergic or similar untoward reaction when administered to a human.

Routes and frequency of administration of the therapeutic compositions described herein, as well as dosage, will vary from individual to individual, and from disease to disease, and may be readily established using standard techniques. In general, the pharmaceutical compositions and vaccines may be administered by injection (e.g., intracutaneous, intramuscular, intravenous or subcutaneous), intranasally (e.g., by aspiration), in pill form (e.g. swallowing, suppository for vaginal or rectal delivery). In certain embodiments, between 1 and 3 doses may be administered over a 6 week period and further booster vaccinations may be given periodically thereafter.

A suitable dose is an amount of an adenovirus vector that, when administered as described above, is capable of promoting a target antigen immune response as described elsewhere herein. In certain embodiments, the immune response is at least 10-50% above the basal (i.e., untreated) level. Such response can be monitored by measuring the target antigen antibodies in a patient or by vaccine-dependent generation of cytolytic effector cells capable of killing influenza infected cells in vitro, or other methods known in the art for monitoring immune responses.

In general, an appropriate dosage and treatment regimen provides the adenovirus vectors in an amount sufficient to provide prophylactic benefit. Protective immune responses may generally be evaluated using standard proliferation, cytotoxicity or cytokine assays, which may be performed using samples obtained from a patient before and after immunization (vaccination).

While one advantage is the capability to administer multiple vaccinations with the same adenovirus vectors, particularly in individuals with preexisting immunity to Ad, the adenoviral vaccines may also be administered as part of a prime and boost regimen. A mixed modality priming and booster inoculation scheme may result in an enhanced immune response.

Thus, one aspect is a method of priming a subject with a plasmid vaccine, such as a plasmid vector comprising a target antigen of interest, by administering the plasmid vaccine at least one time, allowing a predetermined length of time to pass, and then boosting by administering the adenovirus vector. Multiple primings, e.g., 1-3, may be employed, although more may be used. The length of time between priming and boost may vary from about six months to a year, but other time frames may be used.

In certain embodiments, the composition or the replication-defective virus vector further comprises a nucleic acid sequences encoding a costimulatory molecule. In certain embodiments, the costimulatory molecule comprises B7, ICAM-1, LFA-3, or a combination thereof. In particular embodiments, the costimulatory molecule comprises a combination of B7, ICAM-1, and LFA-3. In certain aspects, the composition further comprises a plurality of nucleic acid sequences encoding a plurality of costimulatory molecules positioned in the same replication-defective virus vector. In certain aspects, the composition further comprises a plurality of nucleic acid sequences encoding a plurality of costimulatory molecules positioned in separate replication-defective virus vectors.

In particular aspects, the composition comprises a replication-defective virus vector comprises a nucleic acid sequence encoding an influenza virus target antigen as described herein and further comprises one or more nucleic acid sequences encoding B7, ICAM-1, LFA-3, or a combination thereof.

In some embodiments, the present disclosure provides compositions containing a replication-defective adenovirus vector comprising target antigens at a dose of at least 1×10⁸ viral particles (VPs) and not more than 1×10¹⁰ VPs. In other embodiments, the present disclosure provides compositions containing a replication-defective adenovirus vector comprising target antigens at a dose of at least 1×10⁸ VPs and not more than 5×10¹¹ VPs. In some embodiments, the present disclosure provides compositions containing a replication-defective adenovirus vector comprising target antigens at a dose of at least 1×10⁸ VPs and not more than 1×10¹² VPs. In particular embodiments, the present disclosure provides methods for administration of replication-defective adenovirus vectors at a dose of at least 1×10⁸ VPs and no more than 1×10¹⁰ VPs. In other embodiments, the present disclosure provides methods for administration of replication-defective adenovirus vectors at a dose of at least 1×10⁸ VPs and not more than 5×10¹¹ VPs. In some embodiments, the present disclosure provides methods for administration of replication-defective adenovirus vectors at a dose of at least 1×10⁸ VPs and not more than 1×10¹² VPs.

In further embodiments, vaccine or pharmaceutical compositions described herein may comprise at least, about, or at most 10³, 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, 10²⁰ viral particles or any number or range derivable therefrom. In particular embodiments, vaccine or pharmaceutical compositions described herein may comprise at least, about, or at most 10⁹, 10¹⁰, 10¹¹ viral particles or any number or range derivable therefrom.

In certain embodiments, vaccine or pharmaceutical compositions described herein may be administered at predetermined intervals, such as at an interval of at least, about, or at most once, twice, 3, 4, 5, 6, 7, 8, 9, 10, 11, 12, 13, 14, 15, 16, 17, 18, 19, 20, 30, 40, 50, 100, 120, 130, 140, 150 every hour, day, week, two weeks, three weeks, month, two months, three months, quarter, two quarters, three quarters, year, or decade or any interval or range derivable therefrom.

The various embodiments described above can be combined to provide further embodiments. All of the U.S. patents, U.S. patent application publications, U.S. patent application, foreign patents, foreign patent application and non-patent publications referred to in this specification and/or listed in the Application Data Sheet are incorporated herein by reference, in their entirety to the same extent as if each individual publication, patent, or patent application was specifically and individually indicated to be incorporated by reference.

Aspects of the embodiments can be modified, if necessary to employ concepts of the various patents, application and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

### EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

### Example 1

### Construction of Multiple Influenza Antigen Genes for Insertion into an Ad5 [E1-, E2b-] Vector

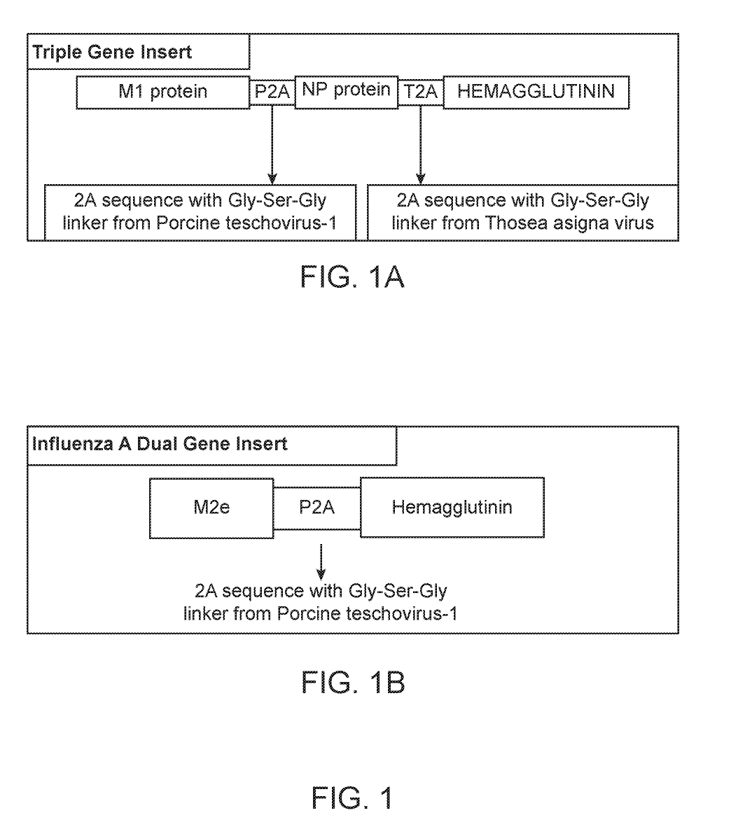

This example describes the construction of multiple influenza antigen genes for insertion into an Ad5 [E1-, E2b-] vector. To produce an Ad5 [E1-, E2b-] containing multiple influenza antigens, the individual influenza antigen gene sequences will be separated by “self-cleaving” 2A peptide derived from Porcine teschovirus-1 and Thosea asigna virus respectively (see FIG. 1A) (de Felipe P and Ryan M Traffic 2004 5(8), 616-26; Holst J et al. Nature Immunol. 2008 9:658-66; Kim J H et al. PloS One, 2011 6(4), e18556. doi:10.1371/journal.pone.0018556). As the 2A peptides are translated on the ribosome, the peptide bond between the final two residues of the 2A peptide is never formed resulting in distinctly expressed proteins in one ribosomal pass. The use of two 2A peptide sequences separating the three genes will result in near stoichiometric expression of the three proteins. Similarly, an Ad5 [E1-, E2b-] containing two antigen gene sequences can be constructed that are separated by a single “self-cleaving” 2A peptide derived from the Porcine teschovirus-1 and Thosea asigna virus, respectively (FIG. 1B).

### Example 2

### Expression of Influenza Proteins after Cellular Infection with Ad5 [E1-, E2b]-M1/NP/HA

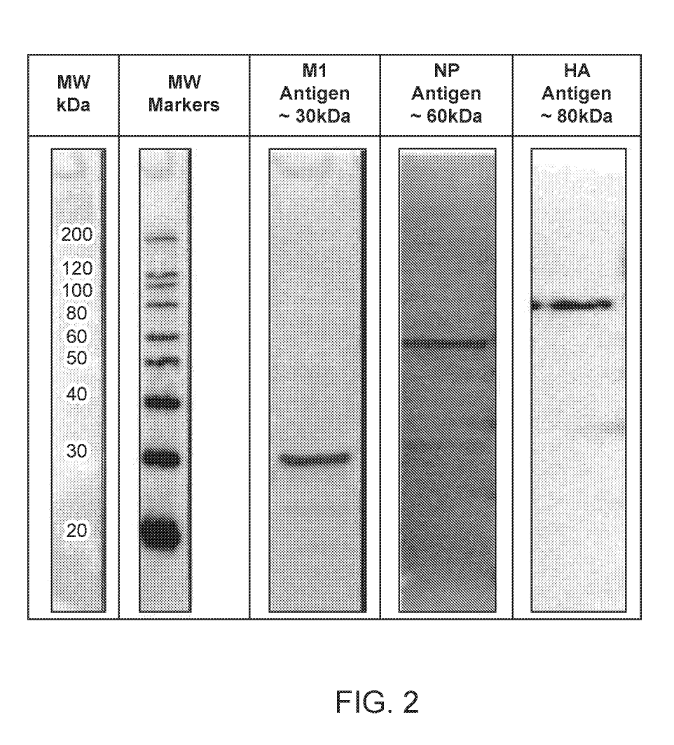

This example describes the expression of influenza proteins after cellular infection with Ad5 [E1-, E2b-]-M1/NP/HA. A549 cells were infected with an Ad5 [E1-, E2b] vector containing matrix 1 (M1) protein, nucleoprotein (NP), and hemagglutinin (HA). Expression of M1, NP, and HA was confirmed by a Western blot. As shown in FIG. 2, an Ad5 [E1-, E2b-]-based platform containing all three (3) hemagglutinin (HA), nucleoprotein (NP), and matrix 1 (M1) protein of gene inserts from influenza A (Ad5 [E1-, E2b-]-M1/NP/HA) has been constructed and produced. As shown in EXAMPLE 3 and EXAMPLE 4, this vaccine was immunogenic in mice inducing HA, NP, and M1 directed immune responses.

### Example 3

### Antibody Response to Ad5 [E1-, E2b-]-M1/NP/HA Immunization

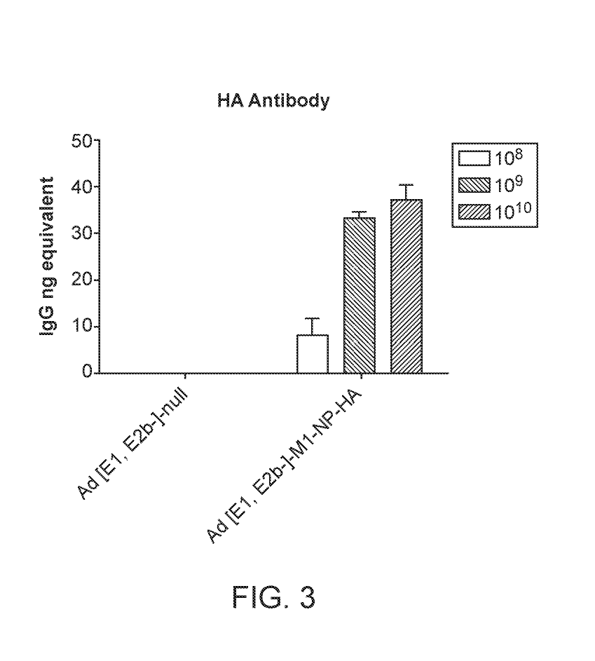

This example describes the antibody response against influenza hemagglutinin (HA) antigen after multiple immunizations with an Ad5 [E1-, E2b-]-vector containing M1 protein, NP protein, and HA protein. Groups of five mice each were immunized two times subcutaneously at weekly intervals with doses of 1×10⁸, 1×10⁹, or 1×10¹⁰ viral particles (VPs) Ad5 [E1-, E2b-]-M1/NP/HA . Control mice were injected with Ad5-null (empty vector) at the same doses. Serum was obtained from individual mice two weeks after the last immunization (vaccination) and assessed for the presence of anti-HA antibodies using a quantitative enzyme-linked immunosorbent assay (ELISA) (Gabitzsch E S et al. Cancer Gene Ther. 2011 18:326-35). As shown in FIG. 3, am anti-HA antibody response was generated in a dose dependent manner in Ad5 [E -, E2b-]-M1/NP/HA immunized mice but not in control mice injected with Ad5-null (an empty vector).

### Example 4

### Cell-mediated Immune (CMI) Responses to Ad5 [E1-, E2b-]-M1/NP/HA Immunization

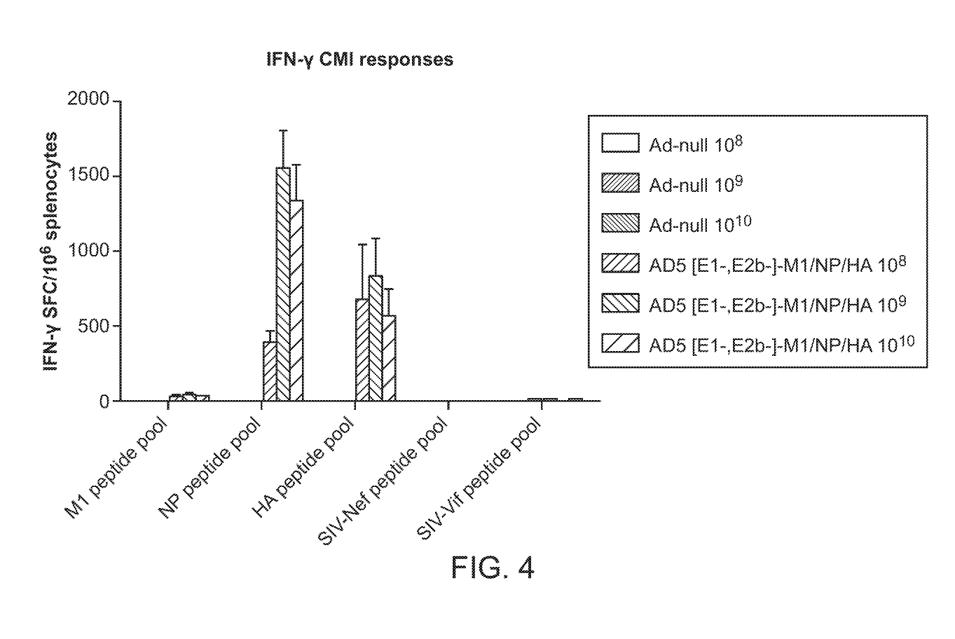

This example describes cell-mediated immune (CMI) responses against influenza antigens after multiple immunizations with an Ad5 [E1-, E2b-]-vector containing M1 protein, NP protein, and HA protein. Groups of five mice each were immunized two times subcutaneously at weekly intervals with doses of 1×10⁸, 1×10⁹, or 1×10¹⁰ VPs Ad5 [E1-, E2b-]-M1/NP/HA. Control mice were injected with Ad5-null (empty vector) at the same doses. Spleens were obtained from individual mice two (2) weeks after the last immunization (vaccination) and assessed for CMI employing ELISpot assays for IFN-y secreting splenocytes (Gabitzsch ES et al. Cancer Immunol Immunother. 2010 59:1131-35; Gabitzsch E S et al. Cancer Gene Ther. 2011 18:326-35; Jones F R et al. Vaccine 2011 29:7020-26). As shown in FIG. 4, CMI responses were generated in Ad5 [E1-, E2b-]-M1/NP/HA immunized mice but not control mice injected with Ad5-null (an empty vector).

### Example 5

### Cytotoxic T Lymphocyte (CTL) Responses to Ad5 [E1-, E2b-]-M1/NP/HA Immunization

This example describes cytotoxic T lymphocyte (CTL) responses to influenza antigens after multiple immunizations with an Ad5 [E1-, E2b-]-vector containing M1 protein, NP protein, and HA protein. CTL response was quantified by measuring granzyme B secretion with an ELISpot assay.

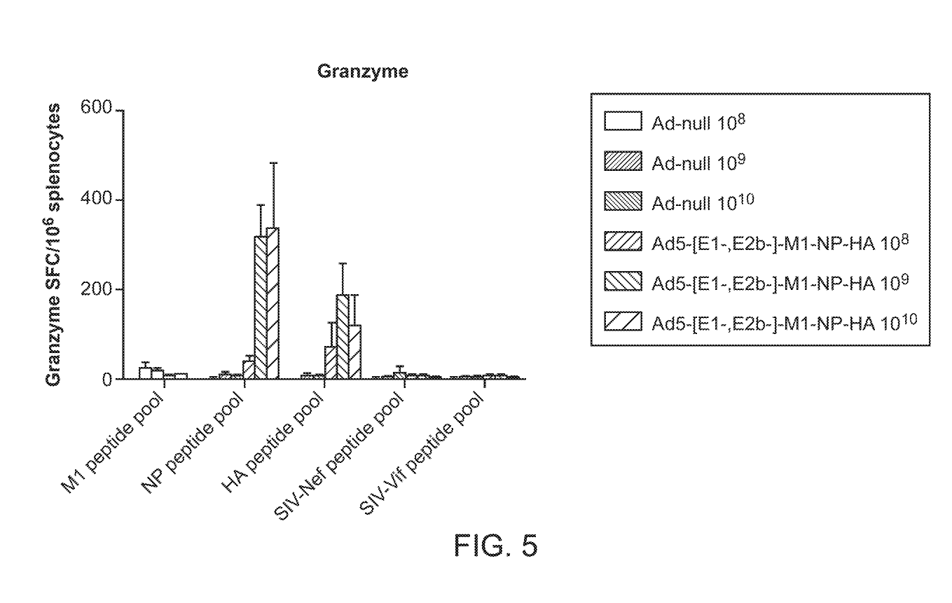

Groups of five mice each were immunized two times subcutaneously at weekly intervals with doses of 1×10⁸, 1×10⁹, or 1×10¹⁰ VPs Ad5 [E1-, E2b-]-M1/NP/HA. Control mice were injected with Ad5-null (empty vector) at the same doses. Spleens were obtained from individual mice two (2) weeks after the last immunization (vaccination) and assessed for cytolytic T lymphocyte (CTL) activity employing ELISpot assays for granzyme B secreting splenocytes. As shown in FIG. 5, CTL responses were generated in Ad5 [E1-, E2b-]-M1/NP/HA immunized mice but not control mice injected with Ad5-null (an empty vector).

### Example 6

### Time-Course Evaluation of CMI Responses to Ad5 [E1-, E2b-]-M1/NP/HA Immunization

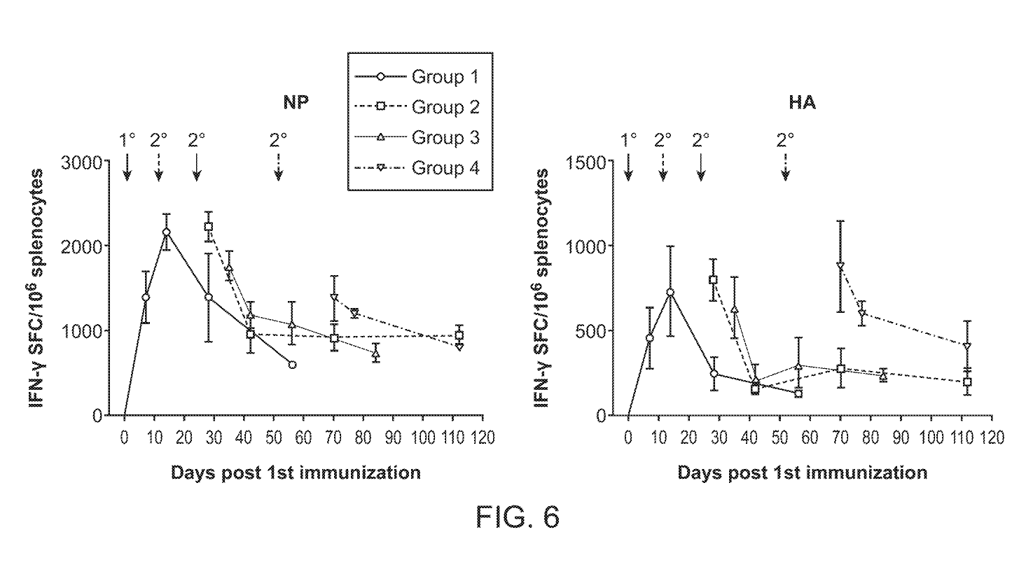

This example describes cell-mediated immune (CMI) responses against influenza antigens at various time points after multiple immunizations with an Ad5 [E1-, E2b-]-vector containing M1 protein, NP protein, and HA protein. Groups of mice each were immunized with 1×10¹⁰ VPs Ad5 [E1-, E2b-]-M1/NP/HA once (Group 1), twice two-weeks apart (Group 2), twice one-month apart (Group 3) and twice two-months apart (Group 4). Spleens were obtained from individual mice at various time points after immunization (vaccination) and assessed for CMI responses against NP and HA by employing ELISpot assays for IFN-γ secreting splenocytes. As shown in FIG. 6, CMI responses were highest generally at one-week after the last immunization and then declined thereafter.

### Example 7

### Administration of Ad5 [E1-, E2b-]-vector Containing m1 Protein, NP Protein, and Hemagglutinin

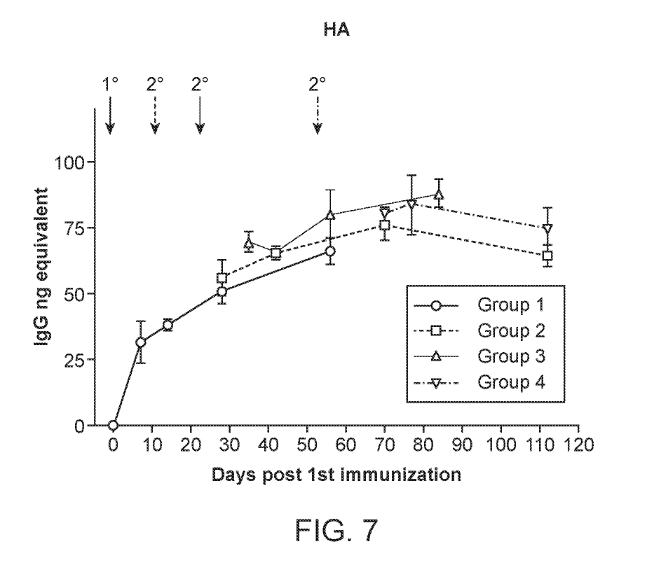

This example describes injections of an Ad5 [E1-, E2b-]-vector containing m1 protein, np protein, and hemagglutinin at various time points to generate an antibody response against influenza hemagglutinin antigen. Groups of mice each were immunized with 1×10¹⁰ VPs Ad5 [E1-, E2b-]-M1/NP/HA once (Group 1), twice 2-weeks apart (Group 2), twice 1-month apart (Group 3) and twice 2-months apart (Group 4). Serum was obtained from individual mice at various time points after immunization (vaccination) and assessed by a quantitative ELISA technique for the presence of anti-HA antibody. As shown in FIG. 7, anti-HA antibody responses were observed to peak at 58 to 85 days after immunization, after which antibody responses were slightly lower.

### Example 8

### Antibody Response to Combination Ad5 [E1-, E2b-]-InfA-HA/M2e and Ad5 [E1-, E2b-]-InfB-HA Immunization

This example describes the antibody response against influenza A (InfA) and influenza B (InfB) antigens after immunization with a combination of an Ad5 [E1-, E2b-]-InfA-HA/M2e vaccine and an Ad5 [E1-, E2b-]-InfB-HA vaccine. The Ad5 [E1-, E2b-]-InfA-HA/M2e was administered at a dose of 1×10¹⁰ viral particles (VPs) and the Ad5 [E1-, E2b-]-InfB-HA vaccine was administered at a dose of 1×10¹⁰ VPs.

Groups of five mice were immunized two times at 2-week intervals with 1×10¹⁰ VPs Ad5 [E1-, E2b-]-null (empty vector) as a negative control, 1×10¹⁰ VPs Ad5 [E1-, E2b-]-Influenza A(InfA)-hemagglutinin (HA)/Matrix 2e (M2e), 1×10¹⁰ VPs Ad5 [E1-, E2b-]-InfB-HA, or a vaccine mixture containing 1×10¹⁰ VPs Ad5 [E1-, E2bd-]-InfA-HA/M2e and 1×10¹⁰ VPs Ad5 [E1-, E2b-]-Influenza B (InfB)-HA. Two weeks after the second immunization, sera from individual mice were analyzed for the presence of antibody to Influenza A-HA or Influenza B-HA using a quantitative ELISA as previously described (Gabitzsch E S et al. Cancer Gene Ther. 2011 18:326-35).

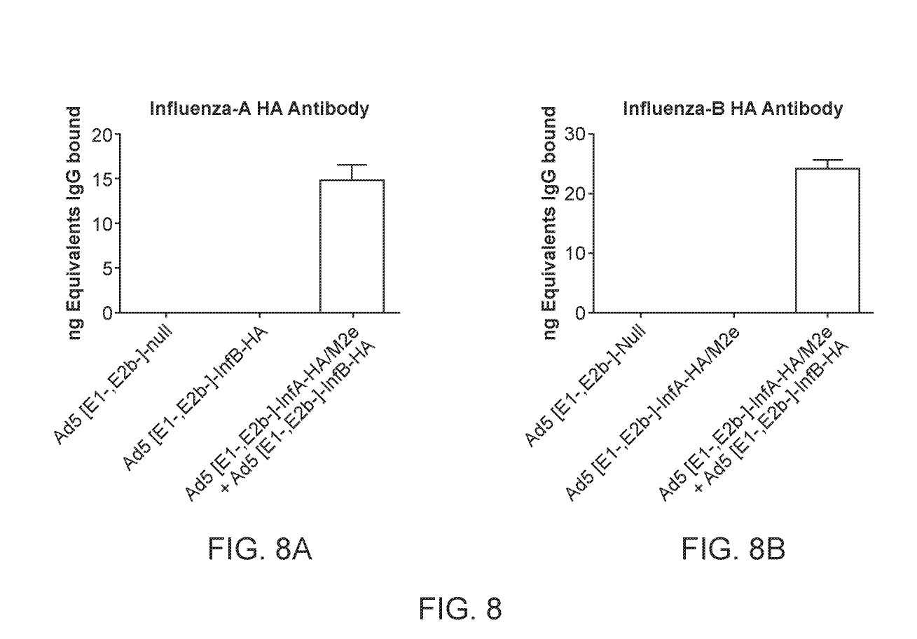

FIG. 8 illustrates quantitation of the Influenza-A or Influenza-B HA antibody response in serum as determined by an enzyme-linked immunosorbent assay (ELISA) after immunization in mice with Ad5 [E1-, E2b-] influenza vaccines. FIG. 8A illustrates quantification of the Influenza-A HA antibody response. FIG. 8B illustrates quantification of the Influenza-B HA antibody response.

Antibody to both Influenza A and B was detected in mice vaccinated with the mixture of Ad5 [E1-, E2b-]-InfA-HA/M2e and Ad5 [E1-, E2b-]-InfB-HA but not in control mice injected with Ad5 [E1-, E2b-]-null. These responses were specific since mice immunized with Ad5 [E1-, E2b-]-InfA-HA/M2e alone did not produce antibody to Influenza B-HA and mice immunized with Ad5 [E1-, E2b-]-InfB alone did not produce antibody to Influenza A-HA.

### Example 9

### Flow Cytometry Analysis of Cell-Mediated Immune (CMI) Responses in Restimulated Splenocytes

This example describes flow cytometry analysis of cell-mediated immune responses after splenocytes derived from mice immunized with a combination of an Ad5 [E1-, E2b-]-InfA-HA/M2e vaccine and an Ad5 [E1-, E2b-]-InfB-HA vaccine are restimulated ex vivo with Influenza HA, Influenza M2, Influenza B HA peptides.

Groups of five mice were immunized two times at two-week intervals with 1×10¹⁰ VPs Ad5 [E1-, E2b-]-null (empty vector), 1×10¹⁰ VPs Ad5 [E1-, E2b-]-Influenza A(InfA)-hemagglutinin (HA)/Matrix 2e (M2e), 1×10¹⁰ VPs Ad5 [E1-, E2b-]-InfB-HA, or a vaccine mixture containing 1×10¹⁰ VPs Ad5 [E1-, E2b-]-InfA-HA/M2e and 1×10¹⁰ VPs Ad5 [E1-, E2b-]-Influenza B (InfB)-HA. Two weeks after the second immunization, spleens from individual mice were analyzed by flow cytometry for CMI activity as evidence by interferon gamma (IFN-γ) expressing CD8+ (A) and/or CD4+ (B) T cells after exposure to specific peptide pools.

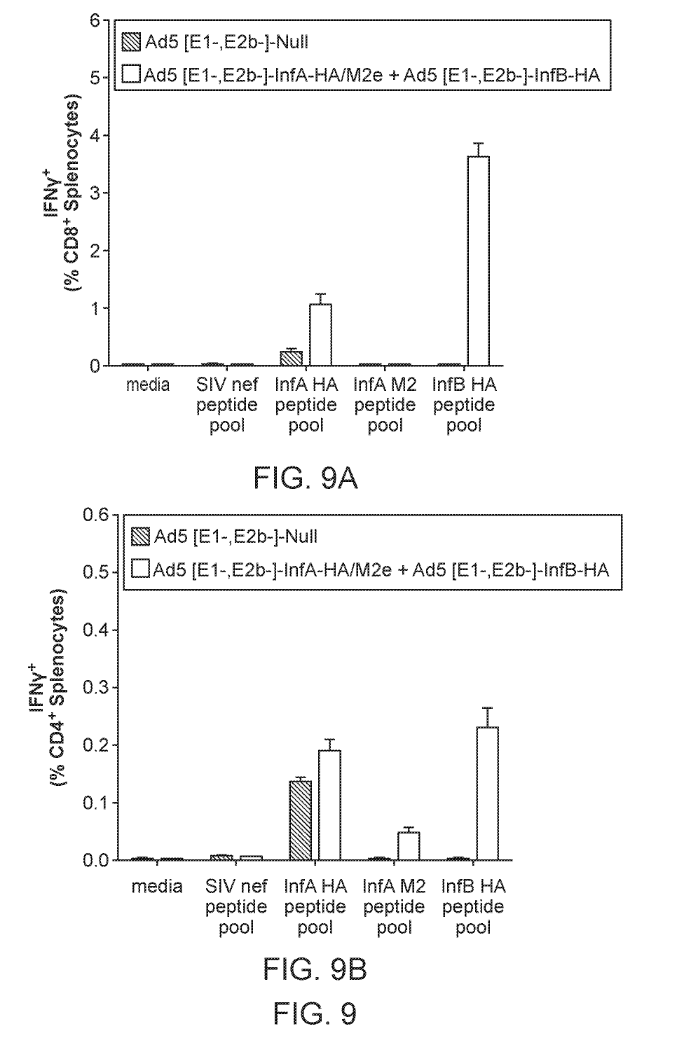

FIG. 9 illustrates the cell-mediated immune response as measured by quantification of IFN-γ-expressing effector T lymphocytes in restimulated splenocytes from mice that have been immunized with a combination of the Ad5 [E1-, E2b-]-InfA-HA/M2e vaccine and the Ad5 [E1-, E2b-]-InfB-HA vaccine. FIG. 9A illustrates the percentage of IFN-γ-expressing CD8+ splenocytes. FIG. 9B illustrates the percentage of IFN-γ-expressing CD4+ splenocytes.

CMI responses to both Influenza A and B were detected in mice vaccinated with the mixture of Ad5 [E1-, E2b-]-InfA-HA/M2e and Ad5 [E1-, E2b-]-InfB-HA but not in control mice injected with Ad5 [E1-, E2b-]-null. Although there was a background response observed against the Influenza HA antigen, a significantly (P<0.05 Mann-Whitney test) higher CMI response was observed in the immunized mice. These responses were specific since T cells from vaccine immunized mice did not produce CMI responses after exposure to media or an irrelevant antigen (SIV-nef) peptide pool.

### Example 10

### ELISpot Analysis of Cell-Mediated Immune (CMI) Responses in Restimulated Splenocytes

This example describes ELISpot analysis of cell-mediated immune responses after splenocytes derived from mice immunized with a combination of an Ad5 [E1-, E2b-]-InfA-HA/M2e vaccine and an Ad5 [E1-, E2b-]-InfB-HA vaccine are restimulated ex vivo with Influenza HA, Influenza M2, Influenza B HA peptides.

Groups of 5 mice were immunized two times at two-week intervals with 1×10¹⁰ VPs Ad5 [E1-, E2b-]-null (empty vector), 1×10¹⁰ VPs Ad5 [E1-, E2b-]-Influenza A(InfA)-hemagglutinin (HA)/Matrix 2e (M2e), 1×10¹⁰ VPs Ad5 [E1-, E2b-]-InfB-HA, or a vaccine mixture containing 1×10¹⁰ VPs Ad5 [E1-, E2b-]-InfA-HA/M2e and 1×10¹⁰ VPs Ad5 [E1-, E2b-]-Influenza B (InfB)-HA. Two weeks after the second immunization, spleens from individual mice were analyzed for CMI activity using ELISpot assays for interferon-gamma (IFN-γ) (A) or IL-2 (B) secreting spot forming cells (SFC) after exposure to specific peptide pools.

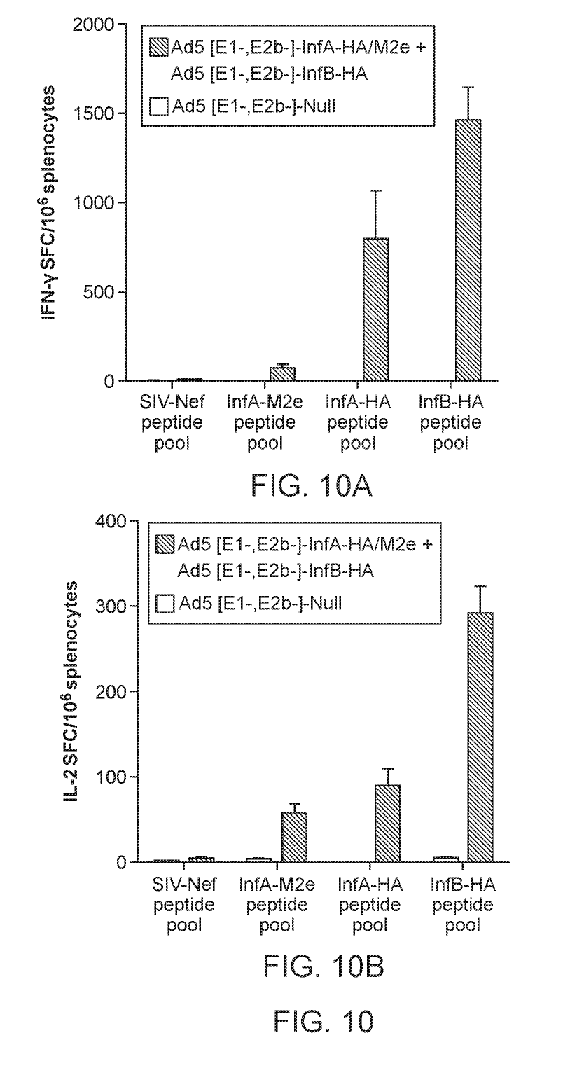

FIG. 10 illustrates the cell-mediated immune response as measured by quantification of cytokine secreting restimulated splenocytes from mice that have been immunized with a combination of the Ad5 [E1-, E2b-]-InfA-HA/M2e vaccine and the Ad5 [E1-, E2b-]-InfB-HA vaccine. FIG. 10A illustrates quantification of IFN-y-secreting splenocytes. FIG. 10B illustrates quantification of IL-2-secreting splenocytes.

CMI responses to both Influenza A and B were detected in mice vaccinated with the mixture of Ad5 [E1-, E2b-]-InfA-HA/M2e and Ad5 [E1-, E2b-]-InfB-HA but not in control mice injected with Ad5 [E1-, E2b-]-null. These responses were specific since spleen cells from vaccine immunized mice did not produce CMI responses after exposure to an irrelevant antigen (SIV-Nef) peptide pool.

### Example 11

### Challenge Study with an Ad5 [E1-, E2b-]-based Influenza Vaccine

This example describes a challenge study with an Ad5 [E1-, E2b-]-based influenza vaccine. Groups of ten mice were immunized two times at two-week intervals with 1×10¹⁰ VPs Ad5 [E1-, E2b-]-null (empty vector) or with a vaccine containing 1×10¹⁰ VPs Ad5 [E1-, E2b-]-M1/NP/InfA-HA as constructed in FIG. 1B of the application. Thirty (30) days after the second immunization, mice were challenged with an H1N1 strain of influenza virus (strain Influenza A/California/07/2009) and assessed for survival after challenge.

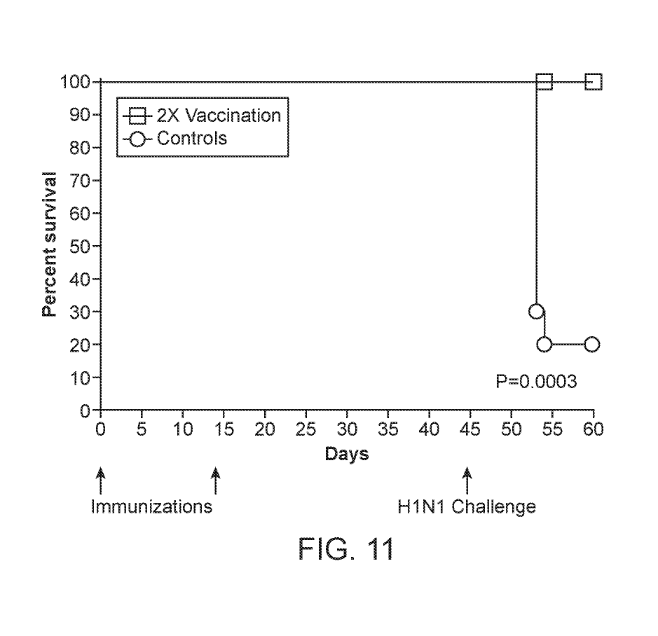

FIG. 11 illustrates a survival curve from the challenge study in mice immunized with an Ad5 [E1-, E2b-]-M1/NP/InfA-HA vaccine as compared with a control (null) vaccine over a period of a 60 days.

Vaccinated mice were afforded complete protection from virus challenge as compared to control mice injected with Ad5 [E1-, E2b-]-null that experienced only 20% survival and this difference was significant (Log Rank Mantel-Cox test, P=0.0003).

While preferred embodiments of the present invention have been shown and described herein, it will be apparent to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A composition comprising:
a replication defective adenovirus vector comprising a deletion in an E2b gene region; and
a nucleic acid sequence encoding an influenza A target antigen and an influenza B target antigen.

2. The composition of claim 1, wherein the influenza A target antigen is a target antigen of an influenza virus A.

3. The composition of claim 1, wherein the influenza A target antigen and the influenza B target antigen are target antigens common to an influenza virus A and an influenza virus B.

4. The composition of any one of claims 1-3, wherein said replication defective adenovirus vector further comprises a deletion in an E1 region.

5. The composition of claim 4, wherein said replication defective adenovirus vector further comprises a deletion in an E3 region.

6. The composition of claim 4, wherein said replication defective adenovirus vector further comprises a deletion in an E4 region.

7. The composition of claim 4, wherein said replication defective adenovirus vector further comprises a deletion in an E3 and an E4 region.

8. The composition of any one of claims 1-7, wherein the influenza A target antigen comprises an antigen of a virus selected from the group consisting of H3N2, H9N1, H1N1, H2N2, H7N7, H1N2, H9N2, H7N2, H7N3, H10N7, and combinations thereof.

9. The composition of any one of claims 1-8, wherein the influenza B target antigen comprises antigens of a virus selected from the influenza B/Yamagata and influenza B/Victoria viruses.

10. The composition of any one of claims 1-9, wherein the influenza A target antigen is an antigen from a protein selected from the group consisting of matrix protein M2, the M2e portion of matrix protein M2, hemagglutinin, hemagglutinin stalk, neuraminidase, nucleoprotein, matrix protein M1, and combinations thereof.

11. The composition of any one of claims 1-10, wherein the influenza B target antigen is an antigen from a protein selected from the group consisting of BM2 protein, hemagglutinin, hemagglutinin stalk, neuraminidase, nucleoprotein, and combinations thereof.

12. The composition of any one of claims 1-11, wherein the deletion comprises a base pair.

13. The composition of claim 12, wherein the deletion comprises at least 20, at least 30, at least 40, at least 50, at least 60, at least 70, at least 80, at least 90, at least 100, at least 110, at least 120, at least 130, at least 140, or at least 150 base pairs.

14. The composition of claim 13, wherein the deletion comprises more than 150, more than 160, more than 170, more than 180, more than 190, more than 200, more than 250, or more than 300 base pairs.

15. The composition of any one of claims 1-14, wherein the adenovirus vector comprises nucleic acids encoding at least one, at least 2, at least 3, at least 4, at least 5, at least 6, at least 7, at least 8, at least 9 or at least 10 influenza A target antigens.

16. The composition of any one of claims 1-15, wherein the adenovirus vector comprises nucleic acids encoding a plurality of influenza A target antigens.

17. The composition of any one of claims 1-16, wherein the adenovirus vector comprises nucleic acids encoding at least one, at least 2, at least 3, at least 4, at least 5, at least 6, at least 7, at least 8, at least 9 or at least 10 influenza B target antigens.

18. The composition of any one of claims 1-17, wherein the adenovirus vector comprises nucleic acids encoding a plurality of influenza B target antigens.

19. The composition of any one of claims 1-18, wherein the adenovirus vector further comprises an element to increase the expression of the influenza A target antigen, the influenza B target antigen, or both.

20. The composition of claim 19, wherein said element comprises at least one element, at least 2 elements, at least 3 elements, at least 4 elements, or at least 5 elements.

21. The composition of claim 19 or 20, wherein said element comprises an internal ribosome binding site.

22. The composition of claim 19 or 20, wherein said element comprises a constitutive promotor.

23. The composition of claim 19 or 20, wherein said element comprises an inducible promotor

24. The composition of claim 19 or 20, wherein said element comprises a transcription enhancer.

25. The composition of claim 24, wherein said transcription enhancer is a Rous sarcoma virus (RSV) enhancer.

26. The composition of any one of claims 19-25, wherein said element does not contain a palindromic sequence.

27. The composition of any one of claims 1-26, wherein the adenovirus vector further comprises nucleic acid sequences that encode proteins that increase the immunogenicity of the influenza A target antigen, the influenza B target antigen, or both.

28. The composition of any one of claims 1-27, wherein the adenovirus vector is not a gutted vector.

29. The composition of any of claims 1-28, wherein the composition or the replication-defective adenovirus vector further comprises a nucleic acid sequences encoding a costimulatory molecule.

30. The composition of claim 29, wherein the costimulatory molecule comprises B7, ICAM-1, LFA-3, or a combination thereof.

31. The composition of claim 30 or 31, wherein the costimulatory molecule comprises a combination of B7, ICAM-1, and LFA-3.

32. The composition of any one of claims 1-31, wherein the adenovirus vector comprises the nucleic acid sequence encoding an influenza A target antigen and an influenza B target antigen.

33. The composition of any one of claims 1-32, wherein the composition comprises at least 1×10⁸ viral particles (VPs) and not more than 5×10¹¹ VPs.

34. The composition of any one of claims 1-32, wherein the composition comprises at least 1×10⁸ viral particles (VPs) and not more than 1×10¹² viral particles VPs.

35. A method of generating an immune response against an influenza A target antigen and an influenza B target antigen in an individual in need thereof, comprising administering to the individual a composition according to any of claims 1-34.

36. A method of generating an immune response against an influenza A target antigen and an influenza B target antigen in an individual comprising administering to the individual a first adenovirus vector comprising:
a replication defective adenovirus vector, wherein the adenovirus vector has a deletion in the E2b region, and
a nucleic acid encoding an influenza A target antigen and an influenza B target antigen; administering to the individual a second adenovirus vector comprising:
(a) a replication defective adenovirus vector, wherein the adenovirus vector has a deletion in the E2b region, and
(b) nucleic acids encoding an influenza A target antigen and an influenza B target antigen; thereby generating an immune response against one or more influenza A and B target antigens.

37. A method of generating an immune response against an influenza A target antigen and an influenza B target antigen in an individual comprising:
(a) administering to the individual a first vector comprising:
(i) a replication defective adenovirus vector, wherein said adenovirus vector has a deletion in the E2b region, and
(ii) a nucleic acid encoding a first influenza A target antigen and a first influenza B target antigen; and
(b) subsequently administering to the individual a second vector comprising:
(i) the replication defective adenovirus vector of step (a), and
(ii) a nucleic acid encoding a second influenza A target antigen and a second influenza B target antigen, wherein the second influenza A target antigen of the second vector is the same or different from the first influenza A target antigen of the first vector, and wherein the second influenza B target antigen of the second vector is the same or different from the first influenza B target antigen of the first vector; thereby generating an immune response against the first target antigen and the second target antigen.

38. A method of generating an immune response against an influenza A target antigen and an influenza B target antigen in an individual comprising: administering to the individual an adenovirus vector comprising a replication defective adenovirus vector, wherein the adenovirus vector has a deletion in the E2b region and nucleic acids encoding an influenza A target antigen and an influenza B target antigen; and re-administering the adenovirus vector at least once to the individual; thereby generating an immune response against the influenza A and B target antigens.

39. A method of constructing a universal influenza vaccine vector comprising inserting nucleic acids encoding an influenza A target antigen and an influenza B target antigen into a replication defective adenovirus vector, wherein the adenovirus vector has a deletion in the E2b region.

40. The method of any one of claims 36-39, wherein the influenza A target antigen comprises an antigen of a virus selected from the group consisting of H3N2, H9N1, H1N1, H2N2, H7N7, H1N2, H9N2, H7N2, H7N3, H10N7, and combinations thereof.

41. The method of any one of claims 36-40, wherein the influenza B target antigen comprises an antigen of a virus selected from the influenza B/Yamagata and influenza B/Victoria viruses.

42. The method of any one of claims 36-41, wherein the influenza A target antigen is an antigen from a protein selected from the group consisting of matrix protein M2, the M2e portion of matrix protein M2, hemagglutinin, hemagglutinin stalk, neuraminidase, nucleoprotein, matrix protein M1, and combinations thereof.

43. The method of any one of claims 36-42, wherein the influenza B target antigen is an antigen from a protein selected from the group consisting of BM2 protein, hemagglutinin, hemagglutinin stalk, neuraminidase, nucleoprotein, and combinations thereof.

44. The method of any one of claims 36-43, wherein the individual has preexisting immunity to adenovirus.

45. The method of any one of claims 36-44, wherein the adenovirus vector is not a gutted vector.

46. The method of any one of claims 36-45, wherein a first vector is not a gutted vector.

47. The method of any one of claims 36-46, wherein a second vector is not a gutted vector.

48. The method of any one of claims 36-47, wherein the first and second adenovirus vectors are not gutted vectors.

49. The method of any one of claims 36-48, wherein the individual has preexisting immunity to adenovirus 5.

50. The method of any one of claims 36-49, wherein the first and second target antigens of the first and the second vectors are derived from the same infectious organism.

51. The method of any one of claims 36-50, wherein the first and second target antigens of the first and the second vectors are derived from different infectious organisms.

52. The method of any one of claims 36-51, wherein the influenza A target antigen and the influenza B target antigen are different target antigens.

53. The method of any one of claims 36-52, wherein the influenza A target antigen is a target antigen of an influenza virus A.

54. The method of any one of claims 36-53, wherein the influenza A target antigen and the influenza B target antigen are target antigens common to an influenza virus A and an influenza virus B.

55. The method of any one of claims 36-54, wherein said replication defective adenovirus vector further comprises a deletion in an E1 region.

56. The method of any one of claims 36-55, wherein said replication defective adenovirus vector further comprises a deletion in an E3 region.

57. The method of any one of claims 36-56, wherein said replication defective adenovirus vector further comprises a deletion in an E4 region.

58. The method of any one of claims 36-57, wherein said replication defective adenovirus vector further comprises a deletion in an E3 and an E4 region.

59. The method of any one of claims 36-58, wherein the deletion comprises a base pair.

60. The method of claim 59, wherein the deletion comprises at least 20, at least 30, at least 40, at least 50, at least 60, at least 70, at least 80, at least 90, at least 100, at least 110, at least 120, at least 130, at least 140, or at least 150 base pairs.

61. The method of claim 60, wherein the deletion comprises more than 150, more than 160, more than 170, more than 180, more than 190, more than 200, more than 250, or more than 300 base pairs.

62. The method of any one of claims 36-61, wherein the adenovirus vector comprises nucleic acid sequences encoding at least one, at least 2, at least 3, at least 4, at least 5, at least 6, at least 7, at least 8, at least 9 or at least 10 influenza A and B target antigens.

63. The method of any one of claims 36-62, wherein the adenovirus vector further comprises an element to increase the expression of the influenza A and influenza B target antigen.

64. The method of claim 63, wherein said element comprise at least one element, at least 2 elements, at least 3 elements, at least 4 elements, or at least 5 elements.

65. The method of claim 63, wherein said element comprises an internal ribosome binding site.

66. The method of claim 63, wherein said element comprises a constitutive promotor.

67. The method of claim 63, wherein said element comprises an inducible promotor.

68. The method of claim 63, wherein said element comprises a transcription enhancer.

69. The method of claim 68, wherein said transcription enhancer is a Rous sarcoma virus (RSV) enhancer.

70. The method of any one of claims 63-69, wherein said element does not contain a palindromic sequence.

71. The method of any one of claims 36-70, wherein the adenovirus vector further comprises a nucleic acid sequence that encodes a polypeptide that increases the immunogenicity of the influenza A target antigen, the influenza B target antigen, or both.

72. The method of any one of claims 36-71, wherein the influenza A target antigen comprises M and the influenza B target antigen comprises BM2.

73. The method of any one of claims 36-72, wherein the influenza A target antigen, the influenza B target antigen, or both comprise hemagglutinin.

74. The method of claim 73, wherein the hemagglutinin comprises an HAI domain.

75. The method of claim 73, wherein herein the hemagglutinin comprises an HA2 domain.

76. The method of claim 73, wherein herein the hemagglutinin comprises a stalk domain.

77. The method of any one of claims 36-76, wherein the influenza A target antigen, the influenza B target antigen, or both comprise a neuraminidase.

78. The method of any one of claims 36-77, wherein the influenza A target antigen, the influenza B target antigen, or both comprise a nucleoprotein (NP).

79. The method of any one of claims 36-78, wherein the influenza A target antigen comprises matrix protein M1.

80. The method of any one of claims 36-79, wherein the influenza A target antigen comprises matrix protein M2.

81. The method of any one of claims 36-80, wherein the influenza A target antigen comprises matrix protein M2e.

82. The method of any one of claims 36-81, wherein the influenza A target antigen, the influenza B target antigen, or both are encoded by a nucleic acid sequence with at least 60%, 70%, 75%, 80%, 85%, 90%, 95%, 97%, 99%, 99.5%, or 100% sequence identity to a sequence encoding a BM2 protein, a hemagglutinin, a hemagglutinin stalk, a neuraminidase, a nucleoprotein, a matrix protein M1, a matrix protein M2 or any combination thereof.

83. The method of any one of claims 36-82, wherein the method comprises administering at least 1×10⁸ viral particles (VPs) and not more than 5×10 VPs.

84. The method of any one of claims 36-82, wherein the method comprises administering at least 1×10⁸ viral particles (VPs) and not more than 1×10¹² viral particles VPs.
